(19) United States
(12) Patent Application Publication
(10) Pub. No.: US 2003/0195205 A1
(43) Pub. Date: Oct. 16, 2003

(54) PDE9 inhibitors for treating cardiovascular disorders

(75) Inventors: Michael Paul DeNinno, Gales Ferry, CT (US); Bernadette Hughes, Sandwich (GB); Mark Ian Kemp, Sandwich (GB); Michael John Palmer, Sandwich (GB); Anthony Wood, Sandwich (GB)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/283,514

(22) Filed: Oct. 30, 2002

Related U.S. Application Data
(60) Provisional application No. 60/350,777, filed on Jan. 22, 2002.
(60) Provisional application No. 60/399,905, filed on Jul. 30, 2002.

(30) Foreign Application Priority Data
Nov. 2, 2001 (GB) 0126395.3
Dec. 21, 2001 (GB) 0130695.0
Jul. 18, 2002 (GB) 0216761.7

(51) Int. Cl. A61K 31/541; A61K 31/5377; A61K 31/519; C07D 487/02

(52) U.S. Cl. 514/228.5; 514/234.5; 514/262.1; 544/60; 544/117; 544/262

(57) ABSTRACT

The invention relates to PDE9 inhibitors for treating cardiovascular disorders. Preferred PDE9 inhibitors are compounds of formula I wherein R¹ is H or C₁₋₆ alkyl, wherein R¹ is attached to either N¹ or N²; R² is C₁₋₆ alkyl optionally substituted by hydroxy or alkoxy; C₃₋₇ cycloalkyl optionally substituted by alkyl, hydroxy or alkoxy; a saturated 5-6-membered heterocycle optionally substituted by alkyl, hydroxy or alkoxy; het1 or Ar¹; R³ is C₁₋₆ alkyl optionally substituted by 1 or 2 groups independently selected from: Ar²; C₃₋₇cycloalkyl optionally substituted by C₁₋₆alkyl; OAr²; SAr²; NHC(O)C₁₋₆ alkyl; het²; xanthene; and naphthalene.

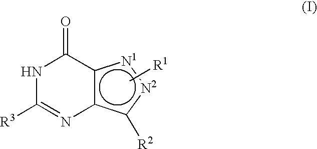

This invention relates to the novel use of cyclic guanylate monophosphate (cGMP)-specific phosphodiesterase type 9 inhibitors (hereinafter referred to as PDE9 inhibitors) for treating a variety of diseases, particularly cardiovascular diseases. In addition, the invention relates to novel PDE9 inhibitors, to processes for preparing them, intermediates used in their preparation, and compositions containing them.

The phosphodiesterase enzyme family hydrolyses cyclic nucleotides cGMP and cyclic adenosine monophosphate (cAMP). cGMP and cAMP are central to the control and regulation of a multitude of cellular events, both physiological and pathophysiological.

D A Fisher et al [J. Biol. Chemistry, vol 273, No 25, 15559-15564 (1998)] identified the PDE9 enzyme as a novel member of the PDE enzyme family that selectively hydrolyses cGMP over cAMP. PDE9 was found to be present in a variety of human tissues, namely the testes, brain, small intestine, skeletal muscle, heart, lung, thymus and spleen. Smooth muscle cells in the human vasculature were not analysed for the presence of PDE9.

We have now found the presence of PDE9 in smooth muscle cells within the human vasculature of a variety of tissues.

Therefore according to a first aspect, the invention provides the use of a PDE9 inhibitor in the manufacture of a medicament for treating or preventing a cardiovascular disorder, disease or condition.

Preferably the cardiovascular disorder, disease or condition is selected from: systemic (or essential) hypertension, pulmonary hypertension (e.g. pulmonary arterial hypertension, pulmonary hypertension of the neonate), congestive heart failure, coronary artery disease, atherosclerosis, stroke, thrombosis, conditions of reduced blood vessel patency (for example post percutaneous transluminal coronary angioplasty), peripheral vascular disease, renal disease (especially that occurring with diabetes), angina (including stable, unstable and variant (Prinzmetal) angina), myocardial ischaemia and any condition where improved blood flow leads to improved end organ function. More preferably the cardiovascular disease is systemic hypertension.

Alternatively the cardiovascular disease may be associated with other conditions, particularly hypertension associated with diabetes.

According to a further aspect there is provided the use of a PDE9 inhibitor in the manufacture of a medicament for treating a condition selected from: male sexual dysfunction (particularly male erectile dysfunction otherwise known as impotence); female sexual dysfunction (FSD) (particularly female hypoactive sexual desire disorder, female sexual arousal disorder, female sexual pain disorder, female orgasmic dysfunction, clitoral dysfunction, dysfunction caused by spinal cord injury and selective serotonin re-uptake inhibitor induced sexual dysfunction), premature labour, dysmenorrhoea, benign prostatic hyperplasia, bladder outlet obstruction, incontinence, nitrate induced tolerance, bronchitis, allergic asthma, chronic asthma, allergic rhinitis, diseases and conditions of the eye (for example glaucoma and optic neuropathy, macular degeneration, elevated intra-ocular pressure, retinal or arterial occlusion), diseases characterised by disorders of gut motility (for example irritable bowel syndrome), pre-eclampsia, Kawasaki's syndrome, nitrate tolerance, multiple sclerosis, neuropathy (including autonomic and peripheral neuropathy), Alzheimer's disease, acute respiratory failure, psoriasis, skin necrosis, cancer, metastasis, baldness, nutcracker oesophagus, anal fissures, haemorrhoids, hypoxic vasoconstriction and stabilisation of blood pressure during haemodialysis.

Without being bound by any theory, we believe that PDE9 inhibitors treat cardiovascular diseases by acting on the nitric oxide/cGMP pathway to mediate relaxation of vascular smooth muscle, thereby causing hypotension, augmenting vascular flow and thus protecting end organ function in disease states where blood flow is compromised.

Preferably (using the assay described hereinafter) the PDE9 inhibitor has a greater than 40% inhibition against PDE9 at a concentration of 1 μM. More preferably the PDE9 inhibitor has an IC50 of less than 500 nM, most preferably an IC50 of less than 50 nM. Preferably the PDE9 inhibitor has a selectivity for PDE9 over PDE1 of greater than 10, preferably greater than 50, most preferably greater than 100.

Preferably the PDE9 inhibitors of the invention are bioavailable when taken orally. Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. Preferably the PDE9 inhibitors have a minimum solubility of 50 μg/ml. Solubility can be determined by standard procedures known in the art such as described in Lipinski C A et al.; Adv. Drug Deliv. Rev. 23(1-3), 3-25, 1997.

Membrane permeability refers to the passage of a compound through the cells of the GIT. Lipophilicity is a key property in predicting this and is defined by in vitro Log D7.4 measurements using organic solvents and buffer. Preferably the PDE9 inhibitors have a Log D7.4 of −2 to +4, more preferably −1 to +3. The Log D can be determined by standard procedures known in the art such as described in Stopher, D and McClean, S; J. Pharm. Pharmacol. 42(2), 144, 1990.

Cell monolayer assays such as Caco2 add substantially to prediction of favourable membrane permeability in the presence of efflux transporters such as P-glycoprotein, so-called Caco2 flux. Preferably, the PDE9 inhibitors have a Caco2 flux of greater than 2×10−6 cms−1, more preferably greater than 5×10−6 cms−1. The Caco2 flux value can be determined by standard procedures known in the art such as described in Artursson, P and Magnusson, C; J. Pharm. Sci, 79(7), 595-600, 1990.

Metabolic stability addresses the ability of the GIT or the liver to metabolise compounds during the absorption process: the first pass effect. Assay systems such as microsomes, hepatocytes etc are predictive of metabolic lability. Preferably the PDE9 inhibitors show metabolic stability in the assay system that is commensurate with an hepatic extraction of less then 0.5. Examples of assay systems and data manipulation are described in Obach, R S; Curr. Opin. Drug Disc. Devel. 4(1), 36-44, 2001 and Shibata, Y et al.; Drug Met. Disp. 28(12), 1518-1523, 2000.

Because of the interplay of the above processes, further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Ward, K W et al.; Drug Met. Disp. 29(1), 82-87, 2001; Berman, J et al.; J. Med. Chem. 40(6), 827-829, 1997 and Han K S and Lee, M G; Drug Met. Disp. 27(2), 221-226, 1999.

A preferred PDE9 inhibitor is a compound of formula I, a pharmaceutically acceptable salt, solvate or prodrug thereof

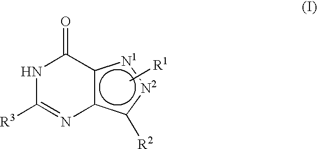

wherein

R¹ is H or C₁₋₆ alkyl, wherein R¹ is attached to either N¹ or N²;

R² is C₁₋₆ alkyl optionally substituted by hydroxy or alkoxy; C₃₋₇ cycloalkyl optionally substituted by alkyl, hydroxy or alkoxy; a saturated 5-6-membered heterocycle (preferably tetrahydrofuran, tetrahydrothiophene, pyrrolidine or piperidine) optionally substituted by alkyl, hydroxy or alkoxy; het¹ or Ar¹;

R³ is C₁₋₆ alkyl optionally substituted by 1 or 2 groups independently selected from: Ar²; C₃₋₇cycloalkyl optionally substituted by C₁₋₆alkyl; OAr²; SAr²; NHC(O)C₁₋₆ alkyl; het²; xanthene; and naphthalene;

wherein Ar¹ and Ar² are independently groups of formula

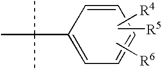

wherein R₄, R₅ and R₆ are independently selected from: hydrogen, halo, phenoxy, phenyl, CF₃, OCF₃, R⁷, SR⁷ and OR⁷, wherein R⁷ is C₁₋₆ alkyl optionally substituted by het³ or by a phenyl group optionally substituted by 1, 2 or 3 groups independently selected from halo, CF₃, OCF₃, C₁₋₆ alkyl and C₁₋₆ alkoxy; or wherein R⁴ and R⁵ combine to form a 3 or 4 atom link, wherein said link may incorporate one or two heteroatoms independently selected from O, S and N; and

wherein het¹, het² and het³, which may be the same or different, are aromatic 5-6 membered heterocycles containing 1, 2 or 3 heteroatoms, independently selected from O, S and N, said heterocycle optionally substituted by 1, 2 or 3 substituents, independently selected from C₁₋₆ alkyl, C₁₋₆alkoxy, halo and phenyl optionally substituted by 1, 2 or 3 groups independently selected from halo and C₁₋₆ alkyl;

with the provisos that when

a) R¹ is attached to N¹, R¹ is C₁₋₃ alkyl and R² is propyl then R³ is not methyl substituted by Ar¹, and

b) R¹ is attached to N¹, R¹ is C₁₋₆ alkyl and R² is methyl then R³ is not C₁₋₄alkyl substituted by Ar¹.

It will be appreciated by the skilled chemist that compounds represented by general formula I cover both compounds of formula Ia and Ib

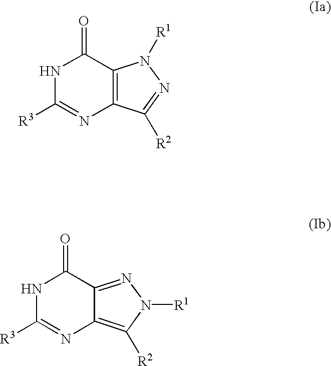

A more preferred PDE9 inhibitor is a compound of formula Ia, a pharmaceutically acceptable salt, solvate or prodrug thereof

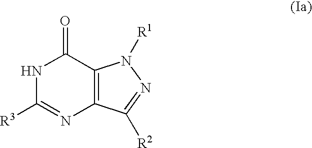

wherein

R¹ is H or C₁₋₆ alkyl;

R² is C₁₋₆ alkyl optionally substituted by hydroxy or alkoxy; C₃₋₇ cycloalkyl optionally substituted by alkyl, hydroxy or alkoxy; a saturated 5-6-membered heterocycle (preferably tetrahydrofuran, tetrahydrothiophene, pyrrolidine or piperidine) optionally substituted by alkyl, hydroxy or alkoxy; het¹ or Ar¹;

R³ is C₁₋₆ alkyl optionally substituted by 1 or 2 groups independently selected from: Ar²; C₃₋₇cycloalkyl optionally substituted by C₁₋₆alkyl; OAr²; SAr²; NHC(O)C₁₋₆ alkyl; het²; xanthene; and naphthalene;

wherein Ar1 and Ar2 are independently groups of formula

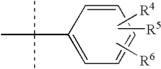

wherein R⁴, R⁵ and R⁶ are independently selected from: hydrogen, halo, phenoxy, phenyl, CF₃, OCF₃, R⁷, SR⁷ and OR⁷, wherein R⁷ is C₁₋₆ alkyl optionally substituted by het3 or by a phenyl group optionally substituted by 1, 2 or 3 groups independently selected from halo, CF₃, OCF₃, C₁₋₆ alkyl and C₁₋₆alkoxy; or wherein R⁴ and R⁵ combine to form a 3 or 4 atom link, wherein said link may incorporate one or two heteroatoms independently selected from O, S and N; and

wherein het¹, het² and het³, which may be the same or different, are aromatic 5-6 membered heterocycles containing 1, 2 or 3 heteroatoms, independently selected from O, S and N, said heterocycle optionally substituted by 1, 2 or 3 substituents, independently selected from C₁₋₆ alkyl, C₁₋₆alkoxy, halo and phenyl optionally substituted by 1, 2 or 3 groups independently selected from halo and C₁₋₆ alkyl;

with the provisos that when,

a) R¹ is C₁₋₃ alkyl and R² is propyl then R³ is not methyl substituted by Ar¹, and

b) R¹ is C₁₋₆ alkyl and R² is methyl then R³ is not C₁₋₄alkyl substituted by Ar¹.

In a further embodiment the invention provides a compound of formula Ia, wherein R¹ is H or C₁₋₆ alkyl; R² is selected from C₁₋₆ alkyl, straight chain or branched chain, C₃₋₇ cycloalkyl and heteroaryl; R³ is C₁₋₆ alkyl, straight chain or branched chain, optionally substituted by 1-2 groups each independently selected from: Ar, C₃₋₇ cycloalkyl, OAr, SAr, NC(O)C₁₋₆ alkyl, heteroaryl, xanthene and naphthalene, wherein:

Ar is a group of formula

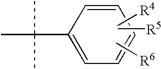

wherein R⁴, R⁵ and R⁶ are each independently selected from: H, halo, OPh, Ph, CF₃, OCF₃, SC₁₋₆ alkyl, C₁₋₆ alkyl, OC₁₋₆ alkyl, said alkyl optionally substituted by a heteroaryl group or by a Ph group, wherein said Ph group is optionally substituted by 1-3 groups selected from halo, CF₃, OCF₃ and C₁₋₆ alkyl; or wherein R⁴ and R⁵ may combine to form a C₁₋₃ alkyl link, wherein said link may optionally incorporate one or two heteroatoms selected from O, S and N, wherein heteroaryl is aromatic 5-6 membered heterocycle containing 1-3 heteroatoms, each independently selected from O, S and N, said heterocycle optionally substituted by 1-3 substituents, each independently selected from C₁₋₆ alkyl, halo and Ph, said Ph optionally substituted by 1-3 groups selected from halo and C₁₋₆ alkyl; with the proviso that when R¹ is —CH₃, R₂ cannot be —CH₂CH₂CH₃; or a pharmaceutically acceptable salt, solvate or prodrug thereof.

Unless otherwise indicated, any alkyl group may be straight or branched and is of 1 to 6 carbon atoms, preferably 1 to 4 and particularly 1 to 3 carbon atoms.

Halo means fluoro, chloro, bromo or iodo.

Preferably R¹ is hydrogen or CH₃. More preferably R¹ is hydrogen.

Preferably R² is C₃₋₄ alkyl, cyclopentyl or pyridyl. More preferably R² is 3-pyridyl.

Preferably R³ is C₁₋₃ alkyl optionally substituted by 1 or 2 groups independently selected from: Ar², C₃₋₇ cycloalkyl optionally substituted by C₁₋₆alkyl and het². More preferably R³ is C₁₋₃ alkyl optionally substituted by Ar². Most preferably R³ is methyl substituted by Ar².

Preferably R⁴, R⁵ and R⁶ are independently selected from: hydrogen, halo, phenoxy, phenyl, CF₃, OCF₃, R⁷, SR⁷, and OR⁷, wherein R⁷ is C₁₋₆alkyl optionally substituted by a het³ group or by a phenyl group optionally substituted by 1, 2 or 3 groups independently selected from halo, CF₃, OCF₃, C₁₋₆ alkyl and C₁₋₆alkoxy; or wherein R⁴ and R⁵ combine to form a 3 atom link wherein said link contains an oxygen atom.

More preferably R⁴, R⁵ and R⁶ are independently selected from hydrogen, halo, CF³, OCF³, phenoxy, and OC₁₋₆ alkyl optionally substituted by phenyl optionally substituted by halo, CF₃, OCF₃ or C₁₋₁₆ alkyl.

Yet more preferably R⁴, R⁵ and R⁶ are independently selected from hydrogen, chloro, OCF₃, CF₃, phenoxy and OC₁₋₆ alkyl substituted by phenyl.

Most preferably, R⁴, R⁵ and R⁶ are independently selected from hydrogen, chloro, OCF₃ and OC₁₋₃ alkyl substituted by phenyl.

Preferably het² is an aromatic 5-6 membered heterocycle containing 1 or 2 nitrogen atoms optionally containing a further heteroatom, said heterocycle being optionally substituted by 1, 2 or 3 substituents, each independently selected from C₁₋₆ alkyl, halo and phenyl optionally substituted by 1, 2 or 3 groups independently selected from halo and C₁₋₆ alkyl.

More preferably het² is an aromatic 5-membered heterocycle containing 1 or 2 nitrogen atoms optionally containing a further heteroatom, said heterocycle being optionally substituted by 1 substituent selected from C₁₋₆ alkyl, halo and phenyl optionally substituted by 1, 2 or 3 groups independently selected from halo and C₁₋₆ alkyl.

Yet more preferably het² is an aromatic 5 membered heterocycle containing 1 or 2 nitrogen atoms optionally containing a further heteroatom and optionally substituted by phenyl optionally substituted by halo.

Most preferably het² is imidazolyl or oxadiazolyl.

Preferred compounds are:

5-(3-chlorobenzyl)-3-isopropyl-1,6-dihydro-pyrazolo[4,3-d]pyrimidin-7-one (compound 1);

3-isopropyl-5-(2-phenoxybenzyl)-1,6-dihydro-pyrazolo[4,3-d]pyrimidin-7-one (compound 52);

3-(3-pyridinyl)-5-(2-benzyloxybenzyl)-1,6-dihydro-pyrazolo[4,3-d]pyrimidin-7-one (compound 138);

3-isopropyl-5-(2-trifluoromethoxybenzyl)-1,6-dihydro-pyrazolo[4,3-d]pyrimidin-7-one (compound 156);

3-cyclopentyl-5-(2-benzyloxybenzyl)-1,6-dihydro-pyrazolo[4,3-d]pyrimidin-7-one (compound 204);

3-(3-pyridinyl)-5-(2-trifluoromethylbenzyl)-1,6-dihydro-pyrazolo[4,3-d]pyrimidin-7-one (compound 215);

3-cyclopentyl-5-(2-trifluoromethoxybenzyl)-1,6-dihydro-pyrazolo[4,3-d]pyrimidin-7-one (compound 258); and

3-(3-pyridinyl)-5-(2-trifluoromethoxybenzyl)-1,6-dihydro-pyrazolo[4,3-d]pyrimidin-7-one (compound 260).

Compounds of formula I as defined hereinabove in the various embodiments of the first aspect are novel. Therefore according to a second aspect, the invention provides a compound of formula I, a pharmaceutically acceptable salt, solvate or prodrug thereof defined hereinabove in the various embodiment of the first aspect.

For the avoidance of doubt, unless otherwise indicated, the term substituted means substituted by one or more defined groups.

For the avoidance of doubt, the term independently means that where more than one substituent is selected from a number of possible substituents, those substituents may be the same or different.

The pharmaceutically acceptable salts of the compounds of formula I which contain a basic centre are, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. Compounds of formula I which contain an acidic centre can provide pharmaceutically acceptable metal salts, in particular non-toxic alkali and alkaline earth metal salts, with bases. Examples include the sodium, potassium, aluminium, calcium, magnesium and zinc salts. Alternatively organic salts can be made, for example the diethanolamine salt. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopaedia of Pharmaceutical Technology, Marcel Dekker Inc, New York 1996, Volume 13, page 453-497.

The pharmaceutically acceptable solvates of the compounds of formula I include hydrates thereof.

Also included within the scope of the invention and various salts of the invention are polymorphs thereof.

Hereinafter, compounds of formula I, their pharmaceutically acceptable salts, their solvates or polymorphs are referred to as “compounds of the invention”.

The compounds of the invention may possess one or more chiral centres and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilising methods known to those skilled in the art. In addition, chiral intermediate compounds may be resolved and used to prepare chiral compounds of the invention.

The compounds of the invention may exist in one or more tautomeric forms. All tautomers and mixtures thereof are included in the scope of the present invention. For example, a claim to 2-hydroxypyridinyl would also cover its tautomeric form, α-pyridonyl.

It will be appreciated by those skilled in the art that certain protected derivatives of compounds of the invention, which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

The invention also includes all suitable isotopic variations of a compound of the invention. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the invention, for example, those in which a radioactive isotope such as 3H or 14C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

Compounds of the invention may be prepared by the following reaction schemes. In the following reaction schemes and hereafter, unless otherwise stated, R¹ to R⁷ are as defined in the first aspect. These processes form further aspects of the invention.

Throughout the specification, general formulae are designated by Roman numerals I, II, III, IV etc. Subsets of these general formulae are defined as Ia, Ib, Ic etc, . . . IVa, IVb, IVc etc.

Compounds of general formula I may be prepared from compounds of general formula II according to reaction scheme 1. Suitable conditions are well known to a man skilled in the art and include base catalysed cyclisation using reagents such as potassium tert-butoxide, sodium hydroxide and potassium carbonate in an alcoholic solvent such as ethanol or isopropanol or an alcohol/water mixture. The reaction may be carried out at a temperature between room temperature and the reflux temperature of the solvent, optionally in the presence of hydrogen peroxide.

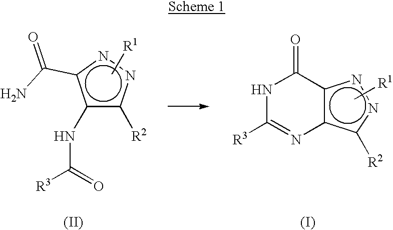

Compounds of general formula II may be prepared according to reaction scheme 2, by reacting compounds of formula IV with compounds of formula III. Such amide bond forming reactions may be carried out under a wide variety of conditions well known to the skilled man. For example, compounds of formula IV may be activated by treatment with an agent such as 1,1-carbonyldiimidazole (CDI) or fluoro-N,N,N′,N′-tetramethylformamidinium hexafluorophosphate (TFFH), or a combination of agents such as azabenzotriazol-1-yloxytris(pyrrolidino)-phosphonium hexafluorophosphate (PyAOP) and 1-hydroxy-7-azabenzotriazole (HOAt), followed by addition of the compound of formula III.

Alternatively, the reaction shown in reaction scheme 2 may be carried out by addition of a peptide coupling agent such as O-(7-azabenzotriazol-1-yl)-N,N,N′,N′-tetramethyluronium hexafluorophosphate (HATU) to a mixture of the compounds of formula III and IV. This reaction is carried out in a suitable solvent such as dichloromethane, pyridine, N,N-dimethylformamide (DMF), N,N-dimethylacetamide (DMA) or 1-methyl-2-pyrrolidinone at a temperature between 0° C. and the reflux temperature of the solvent. The reaction is preferentially carried out by activation of the compound of formula IV with CDI in pyridine under refluxing conditions.

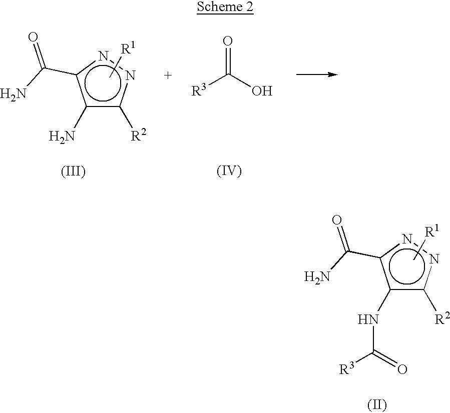

Compounds of general formula III may be prepared from easily obtained starting materials using processes described in the preparative examples hereinafter and described in European Patent EP0463756.

Compounds of general formula IV may be prepared from easily obtained starting materials using processes similar to those described in the preparative examples hereinafter. In addition many compounds of general formula IV are commercially available.

A pharmaceutically acceptable salt of a compound of the formula I may be readily prepared by mixing together solutions of a compound of the formula I and the desired acid or base, as appropriate. The salt may precipitate from solution and be collected by filtration or may be recovered by evaporation of the solvent.

The compounds of the invention can be administered alone but will generally be administered in admixture with a suitable pharmaceutical excipient, diluent or carrier selected with regard to the intended route of administration and standard pharmaceutical practice.

For example, the compounds of the invention can be administered orally, buccally or sublingually in the form of tablets, capsules, multi-particulates, gels, films, ovules, elixirs, solutions or suspensions, which may contain flavouring or colouring agents, for immediate-, delayed-, modified-, sustained-, pulsed- or controlled-release applications. The compounds of the invention may also be administered as fast-dispersing or fast-dissolving dosage forms or in the form of a high energy dispersion or as coated particles. Suitable formulations of the compounds of the invention may be in coated or uncoated form, as desired.

Such solid pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

### GENERAL EXAMPLE

A formulation of the tablet could typically contain between about 0.01 mg and 500 mg of active compound whilst tablet fill weights may range from 50 mg to 1000 mg. An example of a formulation for a 10 mg tablet is illustrated below:

| Ingredient | % w/w |
|---|---|
| Compound of formula I | 10.000* |
| Lactose | 64.125 |
| Starch | 21.375 |
| Croscarmellose sodium | 3.000 |
| Magnesium Stearate | 1.500 |

*Quantity adjusted in accordance with drug activity.

The tablets are manufactured by a standard process, for example, direct compression or a wet or dry granulation process. The tablet cores may be coated with appropriate overcoats.

Solid compositions of a similar type may also be employed as fillers in gelatin or HPMC capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intracavernouslly, intravenously, intra-arterially, intraperitoneally, intrathecally, intraventricularly, intraurethrally, intrasternally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The following dosage levels and other dosage levels herein are for the average human subject having a weight range of about 65 to 70 kg. The skilled person will readily be able to determine the dosage levels required for a subject whose weight falls outside this range, such as children and the elderly.

For oral and parenteral administration to human patients, the daily dosage level of the compounds of the invention will usually be from 0.01 mg to 500 mg (in single or divided doses).

Thus tablets or capsules of the compound of the invention may contain from 0.01 mg to 500 mg (for example 10 mg to 250 mg) of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will appreciate that the compounds of the invention may be taken as a single dose as needed or desired.

The compounds of the invention can also be administered intranasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 μg to 50 mg of a compound of the invention for delivery to the patient. The overall daily dose with an aerosol will be in the range of from 1 μg to 50 mg which may be administered in a single dose or, more usually, in divided doses throughout the day.

Alternatively, the compounds of the invention can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch. They may also be administered by the pulmonary or rectal routes.

They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The present invention additionally comprises the combination of a PDE9 inhibitor, (particularly a compound of formula I as defined in the various embodiments of the first aspect) and one or more additional active agent selected from:

a) a PGI2 prostaglandin, such as prostacyclin or iloprost;

b) an α-adrenergic receptor antagonist compound also known as α-adrenoceptor antagonists, α-receptor antagonists or α-blockers; suitable compounds for use herein include: the α-adrenergic receptor antagonists as described in PCT application WO99/30697 published on Jun. 14, 1998, the disclosures of which relating to α-adrenergic receptor antagonists are incorporated herein by reference and include, selective α1-adrenoceptor antagonists or α2-adrenoceptor antagonists and non-selective adrenoceptor antagonists, suitable α1-adrenoceptor antagonists include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, Recordati 15/2739, SNAP 1069, SNAP 5089, RS17053, SL 89.0591, doxazosin, terazosin, abanoquil and prazosin; α2-blockers from U.S. Pat. No. 6,037,346 [Mar. 14, 2000] dibenarnine, tolazoline, trimazosin and dibenarnine; α-adrenergic receptors as described in U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α2-adrenoceptor antagonists include: clonidine, papaverine, papaverine hydrochloride, optionally in the presence of a cariotonic agent such as pirxamine;

c) an NO-donor (NO-agonist) compound; suitable NO-donor compounds for use herein include organic nitrates, such as mono- di or tri-nitrates or organic nitrate esters including glyceryl trinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP) S-nitroso-N-glutathione (SNO-GLU), N-hydroxy—L-arginine, amylnitrate, linsidomine, linsidomine chlorohydrate, (SIN-1) S-nitroso—N-cysteine, diazenium diolates, (NONOates), 1,5-pentanedinitrate, L-arginene, ginseng, zizphi fructus, molsidomine, Re-2047, nitrosylated maxisylyte derivatives such as NMI-678-11 and NMI-937 as described in published PCT application WO 0012075;

d) a potassium channel opener; suitable potassium channel openers for use herein include nicorandil, cromokalim, levcromakalim, lemakalim, pinacidil, cliazoxide, minoxidil, charybdotoxin, glyburide, 4-aminopyridine, barium chloride;

e) a compound which modulates the action of atrial natruretic factor (also known as atrial naturetic peptide), such as inhibitors of neutral endopeptidase (NEP);

f) a compound which inhibits angiotensin-converting enzyme (ACE) (such as alacepril, alindapril, altiopril, benazepril, benazeprilat, captopril, ceronapril, cilazapril, cilazaprilat, delapril, enalapril, enalaprilat, fosinopril, imidapril, indolapril, libenzapril, lisinopril, moexepril, moveltipril, pentopril, perindopril, quinapril, quinaprilat, ramipril, rentiapril, spirapril, temocapril, teprotide, trandolapril and zofenopril) or a dual ACE/NEP inhibitor, i.e. a compound that inhibits both ACE and NEP (such as, for example, omapatrilat, fasidotril, mixanpril, BMS-189921, MDL-100240 and Z13752A).

g) an angiotensin II receptor blocker (ARB) such as candesartan, eprosartan, irbesartan, losartan, olmesartan, olmesartan medoxomil, saralasin, telmisartan and valsartan.

h) a substrate for NO-synthase, such as L-arginine;

i) a calcium channel blocker such as amlodipine, verapamil, pranidipine, azelnidipine and vatanidipine;

j) an antagonist of endothelin receptors or an inhibitor of endothelin-converting enzyme;

k) a cholesterol lowering agent such as statins. Examples of statins are atorvastatin calcium (Lipitor), cerivastatin sodium (Baycol), fluvastatin sodium (Lescol), lovastatin (Mevacor), pravastatin sodium (Pravachol) and simvastatin (Zocor)

l) an antiplatelet or antithrombotic agent, e.g. tPA, uPA, warfarin, hirudin and other thrombin inhibitors, aspirin, plavix, cilastozol, heparin, thromboplastin activating factor inhibitors;

m) a PDE5 inhibitor (such as 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil); (6R,12aR)-2,3,6,7,12,12a-hexahydro-2-methyl-6-(3,4-methylenedioxyphenyl)pyrazino[2′,1′:6,1]pyrido[3,4-b]indole-1,4-dione (tadalafil, IC-351); 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil);

5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; and

5-(5-acetyl-2-butoxy-3-pyridinyl)-3-ethyl-2-(1-ethyl-3-azetidinyl)-2,6-dihydro-7H-pyrazolo[4,3-d]pyrimidin-7-one); and

n) a beta-blocker, diuretic or aldosterone antagonist.

If a combination of active agents are administered, then they may be administered simultaneously, separately or sequentially.

It is to be appreciated that all references herein to treatment include curative, palliative and prophylactic treatment.

In a further aspect there is provided a method of treating a cardiovascular disorder in a mammal wherein the mammal is treated with an effective amount of a PDE9 inhibitor. The preferred embodiments specified hereinabove for the first aspect extend to this aspect.

The following Examples illustrate the preparation of the compounds of formula I.

### Example 1

Compounds 1 to 126 of formula Ia1 (see Table 3) were prepared, isolated and purified as follows. Each compound was characterised by a) its HPLC retention time (rt) as determined under the conditions described below, and b) by mass spectroscopy also under the conditions described below.

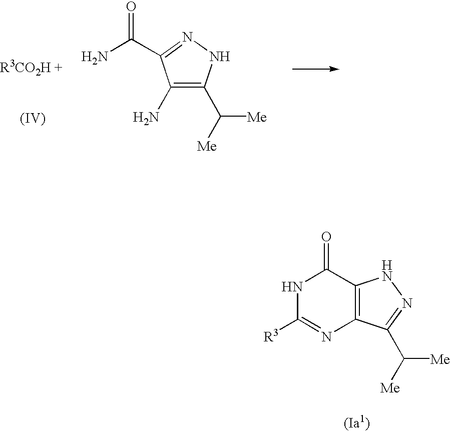

A series of carboxylic acids of general formula IV (where R3 is defined in Table 3) (80 μmol) were each dissolved in a 3.75% solution of triethylamine in dimethylacetamide (400 μl) and added to a 96 well plate. Carbonyldiimidazole (13 mg, 80 μmol) dissolved in pyridine (212 μl) was then added into each well, and the plates were left to stand at room temperature for 2 hours. A solution of the product from Preparation 6 (13.5 mg, 80 μmol) dissolved in dimethylacetamide (100 μl) was then added. The plates were sealed and heated to 70° C. in an oven under nitrogen for 18 hours. The plates were removed and allowed to cool to room temperature over 2 hours. The solvent was removed using a GENEVAC (45° C., 0.15 mbar) over 5.5 hours. A solution of potassium t-butoxide (268 mg, 240 μmol) in isopropyl alcohol (0.5 ml) was added to each well, and the plates were sealed and transferred to an oven at 110° C. under nitrogen for 15 hours. The plates were removed and allowed to cool to room temperature over 2 hours. The solvent was removed using the GENEVAC (45° C., 0.15 mbar) over 5.5 hours, and a solution of p-toluenesulfonic acid (30 mg, 160 μl) in isopropyl alcohol (0.5 ml) was added to each well. The plates were left to stand at room temperature for 18 hours and then the solvent was removed using the GENEVAC (45° C., 0.15 mbar) over 5.5 hours. The residues were dissolved in dimethylsulfoxide: water (0.5 ml per well, 9:1 v/v) and each compound was purified by preparative high pressure liquid chromatography (HPLC) and the desired compound was characterised by liquid chromatography mass spectroscopy (LC-MS).

TABLE 1
Preparative HPLC Conditions:

| Column | Phenomenex Luna C18, 5 μm, 150 × 10 mm i.d. | | | |
|---|---|---|---|---|
| Temperature | Ambient | | | |
| Eluent A | 0.05% diethylamine (aqueous) | | | |
| Eluent B | acetonitrile | | | |
| Sample solvent | 90% dimethylsulfoxide in water | | | |
| Initial pump conditions | A % 95, B % 5, flow 6 ml/min | | | |
| Detection | Gilston 119 uv detector - 225 nm | | | |
| Injection volume | 600 μl | | | |
| Gradient Timetable | Time (min) | A % | B % | Flow (ml/min) |
| | 0.0 | 95 | 5 | 6 |
| | 0.2 | 95 | 5 | 6 |
| | 7.0 | 5 | 95 | 6 |
| | 9.0 | 5 | 95 | 6 |
| | 9.1 | 95 | 5 | 6 |
| | 10.5 | 95 | 5 | 6 |

TABLE 2
LC-MS Conditions

| Column | Phenomenex Luna C18, 5 μm, 30 × 4.6 mm i.d. | | | | |
|---|---|---|---|---|---|
| Temperature | 40° C. | | | | |
| Eluent A | 0.05% diethylamine (aqueous) | | | | |
| Eluent B | acetonitrile | | | | |
| Initial pump conditions | A % 90, B % 10, flow 3 ml/min | | | | |
| Injection volume | 5 ml | | | | |
| Detection | products were detected by both ultraviolet and | | | | |
| | Electron Spray light Scattering (ELSD) | | | | |
| | uv: start range 210 nm, End range 280 nm, Range | | | | |
| | interval 5 nm, threshold 0.1 mAU, peakwidth | | | | |
| | 0.4 min. | | | | |
| | ELSD: Sedere Dedex 55, Temperature: 40° C., | | | | |
| | Gas Flow: 2.3 bar | | | | |
| | | | | Flow | Pressure |
| Gradient Timetable | Time (min) | A % | B % | (ml/min) | (bar) |
| | 0.0 | 90 | 10 | 3 | 400 |
| | 2.2 | 5 | 95 | 3 | 400 |
| | 2.4 | | 95 | 3 | 400 |
| | 2.5 | 90 | 10 | 3 | 400 |

| | |
|---|---|
| Mass spec conditions | Platform: LC |
| | ES+: Cone voltage: 26 v, capillary: 4.08 kv ES−: |
| | Cone voltage: −24 v, capillary: −3.58 kv Blanket |
| | gas: 500 l/min |
| | Temperature: 130° C. |

TABLE 3

| | |
|---|---|
| | (Ia¹) |
| 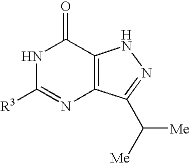 | |

| | | rt | m/z |
|---|---|---|---|
| Cmp | R³ | (min) | [M + H] + |
| 1 | 3-chlorobenzyl | 1.95 | 303 |
| 2 | 4-ethoxybenzyl | 1.85 | 313 |
| 3 | cyclohexylmethyl | 1.92 | 275 |
| 4 | 3-phenoxybenzyl | 2.02 | 361 |
| 5 | 2-chlorobenzyl | 1.85 | 304 |
| 6 | 2-trifluoromethylbenzyl | 1.96 | 337 |
| 7 | 4-chlorobenzyl | 1.91 | 304 |
| 8 | 4-benzyloxybenzyl | 2.05 | 375 |
| 9 | biphenyl-4-ylmethyl | 2.04 | 345 |
| 10 | 2-(2-chlorophenyl)ethyl | 1.93 | 318 |
| 11 | 2,4,6-trifluorobenzyl | 1.89 | 323 |
| 12 | 3,5-bistrifluoromethylbenzyl | 2.07 | 405 |
| 13 | 3-trifluoromethoxybenzyl | 1.97 | 353 |
| 14 | 4-n-butoxybenzyl | 2.08 | 341 |
| 15 | 3-methylbutyl | 1.80 | 249 |
| 16 | 4-methylsulfanylbenzyl | 1.83 | 315 |
| 17 | ethyl | 1.42 | 207 |
| 18 | isobutyl | 1.63 | 235 |
| 19 | 4-methoxybenzyl | 1.77 | 299 |
| 20 | 2,5-dimethylbenzyl | 1.93 | 297 |
| 21 | benzhydryl | 2.07 | 345 |
| 22 | 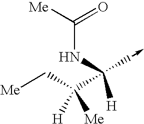 | 1.59 | 306 |
| 23 | 2-fluoro-3-trifluoromethylbenzyl | 1.95 | 355 |
| 24 | 2,4-difluorobenzyl | 1.82 | 305 |
| 25 | 2,3-difluorobenzyl | 1.86 | 305 |
| 26 | 4-fluorobenzyl | 1.80 | 287 |
| 27 | 2-[(2-imidazol-1-yl)-ethoxy]benzyl | 1.44 | 379 |
| 28 | 5-fluoro-2-trifluoromethyl-benzyl | 1.98 | 355 |
| 29 | 2,6-dichlorobenzyl | 1.97 | 338 |
| 30 | 2-chloro-6-methylbenzyl | 2.03 | 334 |
| 31 | 2-methoxybenzyl | 1.76 | 299 |
| 32 | 4-methylphenoxymethyl | 1.92 | 299 |
| 33 | 3,4-difluorobenzyl | 1.83 | 305 |
| 34 | 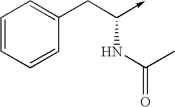 | 1.73 | 340 |
| 35 | 4-methylbenzyl | 1.83 | 283 |
| 36 | 2-(4-methoxyphenyl)-1-phenylethyl | 2.13 | 389 |
| 37 | napthalen-1-ylmethyl | 2.05 | 319 |
| 38 | Cyclopentylmethyl | 1.77 | 261 |
| 39 | 2,6-difluorobenzyl | 1.83 | 305 |
| 40 | 3-methylbenzyl | 1.84 | 283 |
| 41 | 2,4-dimethylbenzyl | 1.99 | 297 |
| 42 | 3-fluorobenzyl | 1.82 | 287 |
| 43 | 2,3,6-trifluorobenzyl | 1.91 | 323 |
| 44 | 4-chlorophenoxymethyl | 1.97 | 320 |
| 45 | 4-phenoxybenzyl | 2.05 | 361 |
| 46 | 2-chloro-6-fluorobenzyl | 1.88 | 322 |
| 47 | 2-benzyloxybenzyl | 2.04 | 375 |
| 48 | 4-methylcyclohexylmethyl | 2.02 | 289 |
| 49 | 1R-1-phenylpropyl | 2.09 | 297 |
| 50 | 2-[3-(4-chlorophenyl)-[1,2,4]oxadiazol-5- | 2.05 | 385 |
| | yl]ethyl | | |
| 51 | n-pentyl | 1.83 | 249 |
| 52 | 2-phenoxybenzyl | 2.03 | 361 |
| 53 | 3,5-dimethylbenzyl | 1.97 | 297 |
| 54 | 4-cyclopentyloxy-3-methoxybenzyl | 1.95 | 383 |
| 55 | 5-napthalen-2-ylmethyl | 1.97 | 319 |
| 56 | 2,5-dichloro-phenylsulfanylmethyl | 2.09 | 370 |
| 57 | 1S-1-phenylethyl | 1.95 | 283 |
| 58 | 2-methylbutyl | 1.78 | 249 |
| 59 | 2,5-difluorobenzyl | 1.86 | 305 |
| 60 | Benzyl | 1.77 | 269 |
| 61 | 4-methylpentyl | 1.94 | 263 |
| 62 | 2-cyclohexylethyl | 2.07 | 289 |
| 63 | 2-chloro-4-fluorobenzyl | 1.92 | 321 |
| 64 | 2-(4-trifluoromethylphenyl)ethyl | 2.03 | 351 |
| 65 | 2-ethoxybenzyl | 1.88 | 313 |
| 66 | Phenoxymethyl | 1.85 | 285 |
| 67 | 3-methoxybenzyl | 1.76 | 299 |
| 68 | 3-trifluoromethylbenzyl | 1.99 | 337 |
| 69 | 4-isopropylbenzyl | 2.04 | 311 |
| 70 | 3,5-difluorobenzyl | 1.89 | 305 |
| 71 | 2,5-dimethoxybenzyl | 1.80 | 329 |
| 72 | 2,3-dimethylbenzyl | 2.04 | 313 |
| 73 | 3,4-dichlorobenzyl | 2.02 | 338 |
| 74 | 4-trifluoromethylbenzyl | 1.93 | 337 |
| 75 | 2-methylbenzyl | 1.85 | 283 |
| 76 | 2-fluorobenzyl | 1.80 | 287 |
| 77 | 4-phenylbutyl | 1.98 | 311 |
| 78 | 2-(3,4-dimethoxyphenyl)ethyl | 1.69 | 343 |
| 79 | 2-(3-methoxyphenyl)ethyl | 1.81 | 313 |
| 80 | 4-ethoxy-3-methoxybenzyl | 1.73 | 343 |
| 81 | 2-(2-methylphenyl)ethyl | 1.92 | 297 |
| 82 | 1-phenoxyethyl | 1.86 | 299 |
| 83 | 2-(3-fluorophenyl)ethyl | 1.85 | 301 |
| 84 | 2,2-diphenylethyl | 2.06 | 359 |
| 85 | 1-methylpropyl | 1.73 | 235 |
| 86 | 3,4-dimethoxybenzyl | 1.65 | 329 |
| 87 | 1-phenoxypropyl | 1.97 | 313 |
| 88 | (3-methoxyphenoxy)methyl | 1.86 | 315 |
| 89 | 2-(4-fluorophenyl)ethyl | 1.85 | 301 |
| 90 | (2-isopropyl-5-methylphenoxy)methyl | 2.19 | 341 |
| 91 | 2-(2,5-dimethoxyphenyl)ethyl | 1.77 | 343 |
| 92 | 3-(4,5-dimethoxyphenyl)propyl | 1.68 | 357 |
| 93 | 2,3-dimethoxybenzyl | 1.84 | 329 |
| 94 | (1,1-diphenyl)ethyl | 2.26 | 359 |
| 95 | 2,3,4-trimethoxybenzyl | 1.80 | 359 |
| 96 | 1-(4-chlorophenoxy)ethyl | 1.97 | 334 |
| 97 | 3,4,5-trimethoxybenzyl | 1.69 | 359 |
| 98 | (3-trifluoromethyl-phenyl)thiomethyl | 2.00 | 369 |
| 99 | 3-pyridylmethyl | 1.28 | 270 |
| 100 | (2-chloro-4-fluorophenyl)thiomethyl | 1.96 | 354 |
| 101 | 1-(4-isobutylphenyl)ethyl | 1.83 | 339 |
| 102 | 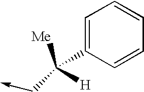 | 1.87 | 297 |
| 103 | (2-methyl-1-phenyl)propyl | 2.13 | 311 |
| 104 | 2-naphthyloxymethyl | 1.98 | 335 |
| 105 | 3-phenylpropyl | 1.87 | 297 |
| 106 | 2-(4-chlorophenyl)ethyl | 1.92 | 318 |
| 107 | 2-(4-methoxyphenyl)ethyl | 1.77 | 313 |
| 108 | (cyclopentyl)(phenyl)methyl | 2.27 | 337 |
| 109 | (2-methoxyphenoxy)methyl | 1.82 | 315 |
| 110 | (1-methyl-1-phenyl)ethyl | 2.08 | 297 |
| 111 | 5-methyl-2-phenyl-4-oxazolylmethyl | 1.95 | 350 |
| 112 | 1-naphthyloxymethyl | 2.06 | 335 |
| 113 | 2-(3,4-dichlorophenyl)ethyl | 2.10 | 352 |
| 114 | 2,4-dimethoxybenzyl | 1.78 | 329 |
| 115 | 3-(4-methoxyphenyl)propyl | 1.86 | 327 |
| 116 | Isopropyl | 1.65 | 221 |
| 117 | 1-[(3-fluoro-4-phenyl)phenyl]ethyl | 2.27 | 377 |
| 118 | (1,2-diphenyl)ethyl | 2.20 | 359 |
| 119 | [2-methyl-4-(phenoxymethyl)]benzyl | 2.05 | 389 |
| 120 | 2-(4-methylphenyl)ethyl | 1.94 | 297 |
| 121 | (3,4-dimethylphenoxy)methyl | 1.98 | 313 |
| 122 | 1-(4-chlorophenyl)ethyl | 2.09 | 318 |
| 123 | [1-methyl-1-(4-chlorophenyl)]ethyl | 2.23 | 332 |
| 124 | 3,5-dimethoxybenzyl | 1.79 | 329 |
| 125 | (2-methyl-1-phenyl)butyl | 2.24 | 325 |
| 126 | 3-phenoxypropyl | 1.85 | 313 |

### Example 2

Compounds 127 to 255 of formula Ia2 (see Table 4) were prepared, isolated and purified by largely analogous procedures to Example 1 by reacting the appropriate carboxylic acid IV with the appropriate compound of formula III, except that the GENEVAC conditions were adjusted to 30 deg, 0.15 mbar for 11 hours in all cases. Each compound was characterised by a) its HPLC retention time (rt) as determined under the conditions described above, and b) by mass spectroscopy under the conditions described above.

TABLE 4

| | | | | (Ia²) |
|---|---|---|---|---|
| 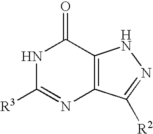 | | | | |
| | | | | m/z |
| | | | | [M + |
| mp | R² | R³ | rt(min) | H] + |
| 127 | 3-pyridyl | 2,4-dichlorobenzyl | 1.24 | 373 |
| 128 | 3-pyridyl | cyclopropylmethyl | 0.92 | 268 |
| 129 | 3-pyridyl | 2,6-difluorobenzyl | 1.10 | 340 |
| 130 | 3-pyridyl | (4-methylcyclohexyl)methyl | 1.46 | 324 |
| 131 | 3-pyridyl | 3-chlorobenzyl | 1.23 | 339 |
| 132 | 3-pyridyl | 2-ethoxybenzyl | 1.24 | 348 |
| 133 | 3-pyridyl | 2-phenoxybenzyl | 1.46 | 396 |
| 134 | 3-pyridyl | 2,3,5-trifluorobenzyl | 1.18 | 358 |
| 135 | 3-pyridyl | 3-fluoro-4-trifluoromethylbenzyl | 1.37 | 390 |
| 136 | 3-pyridyl | 5-fluoro-2-trifluoromethylbenzyl | 1.29 | 390 |
| 137 | 3-pyridyl | 5-bromo-2-methoxybenzyl | 1.30 | 413 |
| 138 | 3-pyridyl | 2-benzyloxybenzyl | 1.42 | 410 |
| 139 | butyl | 2-methylbenzyl | 1.44 | 297 |
| 140 | butyl | 2-methoxybenzyl | 1.36 | 313 |
| 141 | butyl | 2-chlorobenzyl | 1.45 | 318 |
| 142 | butyl | 2-fluorobenzyl | 1.34 | 301 |
| 143 | butyl | 2-chloro-6-fluorobenzyl | 1.47 | 336 |
| 144 | butyl | 2,6-dichlorobenzyl | 1.56 | 352 |
| 145 | butyl | 4-butoxybenzyl | 1.72 | 355 |
| 146 | butyl | cyclopropylmethyl | 1.14 | 247 |
| 147 | butyl | 2,6-difluorobenzyl | 1.38 | 319 |
| 148 | butyl | 2-ethoxybenzyl | 1.48 | 327 |
| 149 | butyl | 3-benzyloxybenzyl | 1.67 | 389 |
| 150 | isopropyl | 2,4,5-trifluorobenzyl | 1.36 | 323 |
| 151 | isopropyl | 2,4-dichlorobenzyl | 1.54 | 338 |
| 152 | isopropyl | 5-bromo-2-methoxybenzyl | 1.46 | 378 |
| 153 | isopropyl | 2,3,6-trichlorobenzyl | 1.59 | 373 |
| 154 | isopropyl | 3-benzyloxybenzyl | 1.59 | 375 |
| 155 | isopropyl | n-propyl | 0.96 | 221 |
| 156 | isopropyl | 2-trifluoromethoxybenzyl | 1.49 | 353 |
| 157 | tert-butyl | 3-chlorobenzyl | 1.61 | 318 |
| 158 | tert-butyl | 5-bromo-2-methoxybenzyl | 1.68 | 392 |
| 159 | isobutyl | 2,4,5-trifluorobenzyl | 1.42 | 337 |
| 160 | isobutyl | 2-methylbenzyl | 1.40 | 297 |
| 161 | isobutyl | cyclopentylmethyl | 1.39 | 275 |
| 162 | isobutyl | isobutyl | 1.20 | 249 |
| 163 | isobutyl | 2-methoxybenzyl | 1.32 | 313 |
| 164 | isobutyl | 2-chlorobenzyl | 1.41 | 318 |
| 165 | isobutyl | 2-fluorobenzyl | 1.31 | 301 |
| 166 | isobutyl | 2-chloro-6-fluorobenzyl | 1.44 | 336 |
| 167 | isobutyl | 2-methylbutyl | 1.35 | 263 |
| 168 | isobutyl | 2-trifluoromethylbenzyl | 1.53 | 351 |
| 169 | isobutyl | 2,4-dichlorobenzyl | 1.61 | 352 |
| 170 | isobutyl | 2,6-dichlorobenzyl | 1.54 | 352 |
| 171 | isobutyl | 4-butoxybenzyl | 1.69 | 355 |
| 172 | isobutyl | cyclopropylmethyl | 1.08 | 247 |
| 173 | isobutyl | 2,6-difluorobenzyl | 1.34 | 319 |
| 174 | isobutyl | 3-chlorobenzyl | 1.44 | 318 |
| 175 | isobutyl | 2-ethoxybenzyl | 1.44 | 327 |
| 176 | isobutyl | 2-phenoxybenzyl | 1.65 | 375 |
| 177 | isobutyl | 2,3,5-trifluorobenzyl | 1.43 | 337 |
| 178 | isobutyl | 5-bromo-2-methoxybenzyl | 1.53 | 392 |
| 179 | isobutyl | 2-benzyloxybenzyl | 1.64 | 389 |
| 180 | isobutyl | 2-(2-imidazol-1-yl-ethoxy)- | 1.12 | 393 |
| | | benzyl | | |
| 181 | Isobutyl | 2,3,6-trichlorobenzyl | 1.66 | 387 |
| 182 | Isobutyl | 3-benzyloxybenzyl | 1.64 | 389 |
| 183 | isobutyl | 2,3-dihydrobenzofuran-5- | 1.25 | 325 |
| | | ylmethyl | | |
| 184 | cyclopentyl | 2,4,5-trifluorobenzyl | 1.51 | 349 |
| 185 | cyclopentyl | 2-methylbenzyl | 1.52 | 309 |
| 186 | cyclopentyl | isobutyl | 1.33 | 261 |
| 187 | cyclopentyl | 2-methoxybenzyl | 1.44 | 325 |
| 188 | cyclopentyl | 2-chlorobenzyl | 1.52 | 330 |
| 189 | cyclopentyl | 2-fluorobenzyl | 1.42 | 313 |
| 190 | cyclopentyl | 2-chloro-6-fluorobenzyl | 1.53 | 347 |
| 191 | cyclopentyl | 2-methylbutyl | 1.49 | 275 |
| 192 | cyclopentyl | 2-trifluoromethylbenzyl | 1.62 | 363 |
| 193 | cyclopentyl | 2,4-dichlorobenzyl | 1.70 | 364 |
| 194 | cyclopentyl | 2,6-dichlorobenzyl | 1.61 | 364 |
| 195 | cyclopentyl | 4-butoxybenzyl | 1.80 | 367 |
| 196 | cyclopentyl | 5-cyclopropylmethyl | 1.22 | 259 |
| 197 | cyclopentyl | 2,6-difluorobenzyl | 1.44 | 331 |
| 198 | cyclopentyl | 2,4,6-trimethoxybenzyl | 1.47 | 385 |
| 199 | cyclopentyl | 3-chlorobenzyl | 1.55 | 330 |
| 200 | cyclopentyl | 2,5-dimethoxybenzyl | 1.41 | 355 |
| 202 | cyclopentyl | 2-phenoxybenzyl | 1.75 | 387 |
| 203 | cyclopentyl | 3-fluoro-4-trifluoromethylbenzyl | 1.67 | 381 |
| 204 | cyclopentyl | 2-benzyloxybenzyl | 1.73 | 401 |
| 205 | cyclopentyl | 2-(2-imidazol-1-yl-ethoxy)benzyl | 1.20 | 405 |
| 206 | cyclopentyl | 3-benzyloxybenzyl | 1.73 | 401 |
| 207 | cyclopentyl | n-propyl | 1.19 | 247 |
| 208 | cyclopentyl | 2,3-dihydro-benzofuran-5- | 1.35 | 337 |
| | | ylmethyl | | |
| 209 | isopropyl | 2-chlorobenzyl | 1.25 | 304 |
| 210 | isopropyl | 2-chloro-6-fluoro-benzyl | 1.25 | 322 |
| 211 | isopropyl | 2,6-dichlorobenzyl | 1.33 | 338 |
| 212 | isopropyl | 2,5-dimethoxybenzyl | 1.15 | 329 |
| 213 | 3-pyridyl | 2-trifluoromethylbenzyl | 1.26 | 372 |
| 214 | 3-pyridyl | 2,6-dichlorobenzyl | 1.24 | 373 |
| 215 | 3-pyridyl | 2,5-dimethoxybenzyl | 1.13 | 364 |
| 216 | 3-pyridyl | n-propyl | 0.89 | 256 |
| 217 | 3-pyridyl | 2,3-dihydrobenzofuran-5- | 1.07 | 346 |
| | | ylmethyl | | |
| 218 | 3-pyridyl | 2,4,6-trimethoxybenzyl | 1.20 | 394 |
| 219 | butyl | isobutyl | 1.24 | 249 |
| 220 | butyl | 2-trifluoromethylbenzyl | 1.55 | 351 |
| 221 | butyl | 2,4-dichlorobenzyl | 1.63 | 352 |
| 222 | butyl | 2,5-dimethoxybenzyl | 1.34 | 343 |
| 223 | butyl | 2-phenoxybenzyl | 1.69 | 375 |
| 224 | butyl | 2-benzyloxybenzyl | 1.67 | 389 |
| 225 | butyl | 2-{[(2- | 1.15 | 393 |
| | | imidazolyl)ethyl]oxy}benzyl | | |
| 226 | butyl | propyl | 1.11 | 235 |
| 227 | butyl | 2,3-dihydrobenzofuran-5- | 1.28 | 325 |
| | | ylmethyl | | |
| 228 | butyl | 2,4,6-trimethylbenzyl | 1.68 | 325 |
| 229 | butyl | 2,4,6-trimethoxybenzyl | 1.40 | 373 |
| 230 | isopropyl | cyclopropylmethyl | 0.99 | 233 |
| 231 | isopropyl | 2,3,5-trifluorobenzyl | 1.36 | 323 |
| 232 | isopropyl | 2,3-dihydrobenzofuran-5- | 1.17 | 311 |
| | | ylmethyl | | |
| 233 | tert-butyl | 2-methoxybenzyl | 1.51 | 313 |
| 234 | tert-butyl | 2-chlorobenzyl | 1.57 | 318 |
| 235 | tert-butyl | 2-fluorobenzyl | 1.47 | 301 |
| 236 | tert-butyl | 2-chloro-6-fluorobenzyl | 1.55 | 336 |
| 237 | tert-butyl | 2-trifluoromethylbenzyl | 1.64 | 351 |
| 238 | tert-butyl | 2,4-dichlorobenzyl | 1.74 | 352 |
| 239 | tert-butyl | 2,6-dichlorobenzyl | 1.63 | 352 |
| 240 | tert-butyl | 4-butoxybenzyl | 1.88 | 355 |
| 241 | tert-butyl | cyclopropylmethyl | 1.33 | 247 |
| 242 | tert-butyl | 2,6-difluorobenzyl | 1.47 | 319 |
| 243 | tert-butyl | 2,5-dimethoxybenzyl | 1.49 | 343 |
| 244 | tert-butyl | 2-ethoxybenzyl | 1.65 | 327 |
| 245 | tert-butyl | 2-phenoxybenzyl | 1.82 | 375 |
| 246 | tert-butyl | 4-CF3-3-fluorobenzyl | 1.72 | 369 |
| 247 | tert-butyl | 2-benzyloxybenzyl | 1.81 | 389 |
| 248 | tert-butyl | 2,3-dihydrobenzofuran-5-ylmethyl | 1.44 | 325 |
| 249 | tert-butyl | 2,4,6-trimethylbenzyl | 1.77 | 325 |
| 250 | tert-butyl | 2,4,6-trimethoxybenzyl | 1.54 | 373 |
| 251 | isobutyl | 2,5-dimethoxybenzyl | 1.31 | 343 |
| 252 | isobutyl | propyl | 1.06 | 235 |
| 253 | isobutyl | 2,4,6-trimethylbenzyl | 1.64 | 325 |
| 254 | isobutyl | 2,4,6-trimethoxybenzyl | 1.38 | 373 |
| 255 | cyclopentyl | 2,4,6-trimethylbenzyl | 1.74 | 337 |

### Example 3

3-Cyclopentyl-5-(2-trifluoromethoxybenzyl)-1,6-dihydro-pyrazolo[4,3-d]pyrimidin-7one (Compound 256)

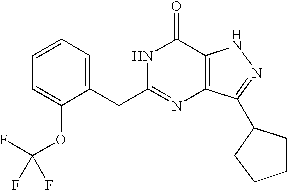

The product from preparation 28 (120 mg, 0.303 mmol) and potassium tert-butoxide (102 mg, 0.909 mmol) were suspended in isopropyl alcohol (5 ml) and the reaction was heated to reflux under nitrogen for 18 h. The reaction mixture was concentrated under reduced pressure and the residue partitioned between ethyl acetate (20 ml) and water (20 ml). The aqueous phase was removed, acidified to pH2 with 2N HCl, and extracted with ethyl acetate (2×15 ml). The combined organic extracts were washed with saturated sodium carbonate solution (3×10 ml), dried over MgSO4, concentrated under reduced pressure and the residue was purified by flash column chromatography on silica gel eluting with dichloromethane: methanol (95:5, by volume) to give the title product (21 mg) as an off-white solid, ¹H NMR (400 MHz, DMSO-d6): δ=7.36-7.41 (2H, m), 7.29-7.36 (2H, m), 3.97-4.03 (2H, brs), 2.39-2.45 (1H, m, partially masked by solvent), 1.82-1.94 (2H, m), 1.66-1.79 (2H, m), 1.58-1.65 (2H, m), 1.49-1.58 (2H, m) ppm; LRMS (electrospray): m/z [M−H]+377.

Compounds 257 to 261 of formula Ia2 were prepared by methods analogous to Example 3 from the starting materials indicated in Table 5 below.

TABLE 5

| | |
|---|---|
| | (Ia²) |
| 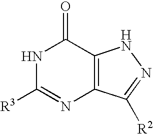 | |

| | | | Starting | |
|---|---|---|---|---|
| mp | R² | R³ | Material | Data |
| 257 | isobutyl | 2-trifluoromethoxy-benzyl | Prep 29 | ¹H NMR(400MHz, CDCl₃): |
| | | | | δ 8.64-8.74(1H, brs), 7.22-7.41 |
| | | | | (4H, m, partially masked by |
| | | | | solvent), 4.15(2H, s), 2.79-2.84 |
| | | | | (2H, d), 2.13-2.23(1H, m), 0.92- |
| | | | | 1.00(6H, d) ppm; LRMS |
| | | | | (electrospray) : m/z [M + H] + 367, |
| | | | | [M − H] + 365. |
| 258 | 3-pyridyl | 2-trifluoromethoxy-benzyl | Prep 30 | ¹H NMR(400MHz, |
| | | | | CD₃OD): δ 9.34(1H, brs), 8.57-8.61 |
| | | | | (1H, d), 8.43-8.48(1H, m), 7.32- |
| | | | | 7.47(5H, m), 4.18(2H, s) ppm; |
| | | | | LRMS(electrospray): m/z [M − H] + |
| | | | | 386. |
| 259 | isopropyl | 2-(3-chlorobenzyl-oxy)benzyl | Prep 32 | ¹H NMR(400MHz, |
| | | | | CD₃OD): δ 7.2(m, 6H); 7.0(d, 1H, J |
| | | | | 8.3Hz); 6.9(m, 1H); 5.02(s, 2H); |
| | | | | 4.0(s, 2H); 3.3(m, 1H); 1.3(d, |
| | | | | 6H, J 7.0Hz); LCMS: m/z [M + H] + |
| | | | | 409. |
| 260 | isopropyl | 2-(4-chlorobenzyl-oxy)benzyl | Prep 33 | ¹H NMR(400MHz, d6 |
| | | | | acetone): δ 7.45(d, 2H, J 8.3Hz); |
| | | | | 7.35(d, 2H, J 8.3Hz); 7.24(m, |
| | | | | 2H); 7.08(d, 1H, J 7.5Hz); 6.94(t, |
| | | | | 1H, J 7.5Hz); 5.02(s, 2H); 4.08 |
| | | | | (s, 2H); 3.26(m, 1H); 1.35(d, 6H, |
| | | | | J 6.6Hz); LCMS: m/z [M + H] + 409. |
| 261 | isopropyl | 2-benzyloxy-5-chlorobenzyl | Prep 35 | 1H NMR (400MHz, d6 |
| | | | | acetone): δ 7.4-7.24(m, 7H); 7.1 |
| | | | | (d, 1H, J 8.7Hz); 5.19(s, 2H); |
| | | | | 4.05(s, 2H); 3.22(m, 1H); 2.8 |
| | | | | (bm, 2H); 1.32(d, 6H, J 6.6Hz). |
| | | | | LCMS: m/z [M + H] + 409. |

### Example 4

### 5-(3-Chlorobenzyl)-3-isopropyl-2-methyl-2,6-dihydro-7H-pyrazolo[4,3-d]pyrimidin-7-one (Compound 262)

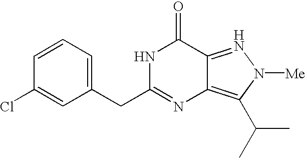

To a mixture of the product from Preparation 31 (0.2 g) in isopropyl alcohol (6 ml) was added potassium tert-butoxide (2.2 g) and stirred at 85° C. for 24 hours and then at room temperature for 3 days. The resulting heterogeneous mixture was concentrated in vacuo. Water (10 ml) was added to the residue followed by 3 drops of concentrated hydrochloric acid. The resulting precipitate was taken up in ethyl acetate (150 ml) and washed with water (×2). The organic extract was dried (MgSO4) and concentrated to give a solid which was purified by column chromatography using silica gel eluting with a solvent gradient of dichloromethane:methanol (100:0 changing to 99:1 changing to 98:2) to give the title product; 1H NMR (400 MHz, CD₃OD): δ=7.39 (1H, s), 7.28 (1H, s), 7.12 (2H, m), 4.01 (3H, s), 3.96 (2H, s), 3.26 (1H, m), 1.45 (6H, d).

### Example 5

### 5-(2-Trifluoromethoxybenzyl)-3-phenyl-1,6-dihydro-7H-pyrazolo[4,3-d]pyrimidin-7-one (Compound 263)

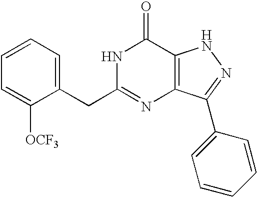

Palladium tetrakis triphenylphosphine (22 mg, 5 mole %) was added to a nitrogen-purged solution of the product from preparation 36 (164 mg, 0.376 mmol), phenyl boronic acid (69 mg, 0.56 mmol), sodium carbonate (119 mg, 1.13 mmol as a solution in 0.8 ml water) in ethylene glycol dimethyl ether (3 ml). The mixture was heated at 83° C. for 18 h. On cooling, the mixture was diluted with ethyl acetate/tetrahydrofuran and washed with brine, dried (MgSO4), filtered and concentrated in vacuo. The residue was purified by preparative HPLC (acetonitrile-water gradient) to afford the title product (15 mg) as a colourless solid. 1H NMR (400 MHz, CD₃OD): δ=8.2 (bs, 2H); 7.4 (m, 9H); 4.1 (s, 2H); LCMS: m/z [M+H]+387.

The following Preparations describe the preparation of certain intermediates used in the preceding Examples.

### Preparation 1

### 5-Methyl-2,4-dioxo-hexanoic Acid Ethyl Ester

Sodium pellets (3.39 g, 148 mmol) were dissolved in ethanol (100 ml) under nitrogen at room temperature and a solution of diethyloxalate (20 ml, 147 mmol) in 3-methyl-2-butanone (18.9 ml, 177 mmol) was added dropwise at room temperature over 30 min. The reaction was diluted with ethanol (100 ml), heated to 60° C. and stirred at this temperature for 2 h. After cooling to room temperature the reaction was poured onto ice-cold 2N HCl (200 ml) and extracted with diethylether (300 ml) and ethyl acetate (300 ml). The combined organic extracts were dried over MgSO4, concentrated under reduced pressure and the residue was purified by flash column chromatography on silica gel eluting with a solvent gradient of pentane:ethyl acetate (99:1 changing to 95:5, by volume) to give the title product (23.8 g) as a yellow oil;

1H NMR (400 MHz, CDCl₃): δ=14.40-14.80 (1H, brs), 6.40 (1H, s), 4.30-4.39 (2H, quart), 2.60-2.71 (1H, quin), 1.35-1.40 (3H, t), 1.15-1.20 (6H, d) ppm; LRMS (electrospray): m/z [M−H]+185.

### Preparation 2

### 5-Isopropyl-1H-pyrazol-3-carboxylic Acid Ethyl Ester

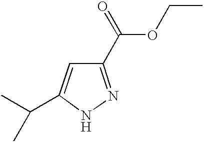

Hydrazine hydrate (6.6 ml, 134 mmol) was added to a solution of the product from Preparation 1 (23.8 g, 188 mmol) in ethanol (100 ml) at room temperature under nitrogen. The reaction was allowed to proceed at room temperature for 18 h, and the solvent was removed under reduced pressure. The residue was partitioned between dichloromethane (300 ml) and water (300 ml) and the aqueous phase was removed. The organic phase was washed with water (2×200 ml), dried over MgSO₄ and concentrated under reduced pressure. The residue was purified by flash column chromatography on silica gel eluting with a solvent gradient of pentane:ethyl acetate (4:1 changing to 2:1, by volume) to give the title product (18.9 g) as a white solid; ¹H NMR (400 MHz, CDCl₃): δ=10.80-10.95 (1H, brs), 6.61 (1H, s), 4.33-4.40 (2H, quart), 2.98-3.08 (1H, quin), 1.35-1.41 (3H, t), 1.24-1.32 (6H, d) ppm; LRMS (electrospray): m/z [M−H]+181.

### Preparation 3

### 5-Isopropyl-1H-pyrazol-3-carboxylic Acid

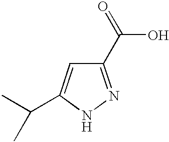

The product from preparation 2 (18.9 g, 104 mmol) and 1M NaOH solution (260 ml, 259 mmol) were dissolved in 1,4-dioxan (300 ml), the reaction was heated to 50° C. under nitrogen and stirred for 3 h. The reaction mixture was cooled, adjusted to pH 2 using concentrated hydrochloric acid and the solvent was removed under reduced pressure. The residual solid was azeotroped with toluene (2×30 ml), dissolved in ethyl acetate (500 ml) and washed with water (200 ml). The aqueous phase was removed, extracted with ethyl acetate (2×200 ml) and the combined organic extracts were dried over MgSO₄. The solvent was removed under reduced pressure and the residue was azeotroped with dichloromethane (2×50 ml) to give the title product (14.7 g) as a white solid; ¹H NMR (400 MHz, DMSO-D6): δ=12.50-13.30 (2H, brs), 6.42 (1H, s), 2.84-2.94 (1H, quin), 1.15-1.19 (6H, d) ppm; LRMS (electrospray): m/z [M−H]+153.

### Preparation 4

### 5-Isopropyl-4-nitro-1H-pyrazol-3-carboxylic Acid

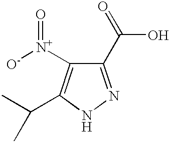

The product from preparation 3 (5 g, 32.5 mmol) was added portionwise to concentrated sulfuric acid (25 ml) at room temperature with stirring. The reaction mixture was then heated to 60° C. and concentrated nitric acid (70%, 6 ml, 90 mmol) was added dropwise whilst keeping the temperature at 60° C. The reaction was stirred at 60° C. for 3 h, cooled to room temperature and then poured onto 50 ml of ice with stirring. After 15 min the white precipitate was isolated by filtration, washed with water and dried under reduced pressure to give the title product (5.2 g) as a white solid; ¹H NMR (400 MHz, DMSO-D6): δ=13.86-13.93 (1H, brs), 13.50-13.80 (1H, brs), 3.39-3.52 (1H, m), 1.18-1.30 (6H, d) ppm; LRMS (electrospray): m/z [M−H]+198.

### Preparation 5

### 5-Isopropyl-4-nitro-1H-pyrazol-3-carboxylic Acid Amide

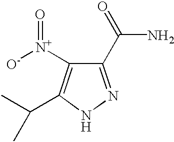

Oxalyl chloride (6.8 ml, 77.6 mmol) was added dropwise to a suspension of the product from preparation 4 (5.15 g, 25.9 mmol) in dichloromethane (80 ml) containing dimethylformamide (0.1 ml) under nitrogen at 0° C. The reaction was stirred at 0° C. for 1 h, allowed to warm to room temperature and stirred for a further 2 h. The solvent was removed under reduced pressure, the residue was dissolved in toluene (100 ml) and ammonia gas was bubbled into the solution for 2 h. The reaction was stirred under nitrogen at room temperature for 18 h, concentrated under reduced pressure and the residue was dissolved in hot methanol (300 ml). The resultant precipitate was filtered and the filtrate was concentrated under reduced pressure. The residue was azeotroped with water (300 ml), concentrated to approximately 80 ml under reduced pressure and the precipitate was isolated by filtration. The filtrate was washed with water and dried in vacuo to give the title product (3.1 g) as an orange solid; ¹H NMR (400 MHz, DMSO-D6): δ=7.94-7.99 (1H, brs), 7.68-7.72 (1H, brs), 3.45-3.55 (1H, m), 1.24-1.30 (6H, d) ppm; LRMS (electrospray): m/z [M+Na]+221, [M−H]+197.

### Preparation 6

### 4-Amino-5-isopropyl-1H-pyrazol-3-carboxylic Acid Amide

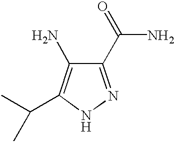

The product from preparation 5 (3 g, 15.1 mmol) and 10% palladium on carbon (500 mg) in ethanol (30 ml) were stirred under hydrogen (50 psi) at room temperature for 18 h. The reaction mixture was filtered and the solid was washed with methanol (50 ml), dichloromethane (50 ml), ethanol (50 ml) and ethyl acetate (50 ml). The filtrate was concentrated under reduced pressure and the residue was purified by flash column chromatography on silica gel eluting with dichloromethane:methanol (9:1, by volume) to give the title product (2.6 g) as an off-white solid; ¹H NMR (400 MHz, DMSO-D6): δ=12.20-12.30 (1H, brs), 7.02-7.14 (1H, brs), 6.85-6.95 (1H, brs), 4.30-4.46 (2H, brs), 2.90-3.00 (1H, m), 1.15-1.21 (6H, d) ppm; LRMS (electrospray): m/z [M−H]+167, [2M−H]+335; Anal. Found C, 49.86; H, 7.21; N, 33.07. C7H12N4O requires C, 49.99; H, 7.19; N, 33.31%.

Preparations 7 to 10 of general formula IIIa were prepared by methods analogous to Preparations 1 to 6 from the starting materials indicated in Table 6.

TABLE 6

| | |
|---|---|
| | (IIIa) |
| 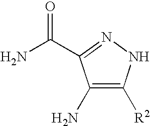 | |

| Prep | R² | Starting Material |
|---|---|---|
| 7 | butyl | hexan-2-one |
| 8 | tert-butyl | tert-butylmethyl ketone |
| 9 | isobutyl | isobutyl methyl ketone |
| 10 | cyclopentyl | cyclopentylethanone |

### Preparation 11

### 4-Amino-5-isopropyl-1-methyl-1H-pyrazole-3-carboxamide

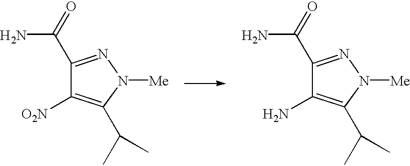

The product from preparation 12 in ethanol was hydrogenated at 50° C. and 50 p.s.i. for 2 hours in the presence of 10% paladium on charcoal. The reaction mixture was filtered, concentrated in vacuo, and azeotroped with dichloromethane to give the title product as a solid (0.16 g); ¹H NMR (400 MHz, CDCl₃): δ=6.99 (1H, br.s), 6.86 (1H, br.s), 4.41 (2H, br.s), 3.66 (3H, s), 3.02 (1H, m), 1.19 (6H, d).

### Preparation 12

### 5-Isopropyl-1-methyl-4-nitro-1H-pyrazole-3-carboxamide

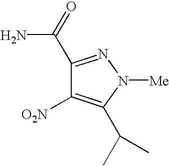

To a mixture of the product from preparation 5 (150 mg) in acetonitrile (6 ml) was added ceasium carbonate (107 mg) followed by iodomethane (40 μl). The mixture was heated at 77° C. overnight. The mixture was concentrated in vacuo and the residue taken up in ethyl acetate (200 ml) and washed with brine. The organic extracts were dried (MgSO₄), the solvent was removed and the residue purified by chromatography using silica gel eluting with pentane:ethyl acetate (100:0 to 15:1 to 1:1); 1H NMR (400 MHz, CDCl3): δ=6.50 (1H, br.s), 5.65 (1H, br.s), 3.89 (3H, s), 3.43 (1H, m), 1.36 (6H, d).

### Preparation 13

### 4-Amino-5-(3-pyridyl)-1H-pyrazole-3-carboxamide

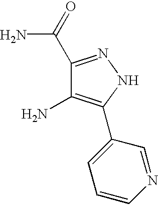

A solution of the product from preparation 14 (1 g) in 0.88M ammonia (100 ml) were heated at 100° C. in a bomb overnight. The mixture was concentrated in vacuo and the residue was purified by chromatography using silica gel eluting with dichloromethane:methanol (9:1) to give the title product (709 mg).

### Preparation 14

### Ethyl 4-amino-5-(3-pyridyl)-1H-pyrazole-3-carboxylate

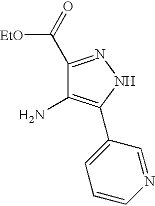

To an ice-cooled solution of sodium ethoxide (34 ml, 21% w/w) in ethanol (50 ml) was added 3-(cyanomethyl)pyridine (10 ml) dropwise over 30 minutes. The reaction mixture was stirred for 30 minutes at 0° C. and diazoacetic acid (9.9 ml) was added dropwise over 15 minutes. The reaction mixture was allowed to warm to room temperature and stirred overnight. Water was added and the solution was neutralised with carbon dioxide. On extracting the mixture with ethyl acetate and dichloromethane (approx 1000 ml), a solid precipitated. Filtration of the solid gave the title product (13.6 g).

### Preparation 15

### 4-Amino-1H-pyrazol-3-carboxylic Acid Amide

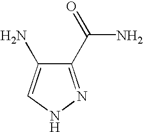

The title product was prepared by an analogous method to Preparation 6 starting from the product of Preparation 16.

### Preparation 16

### 4-Nitro-1H-pyrazol-3-carboxylic Acid Amide

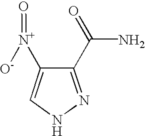

The title product was prepared by an analogous method to Preparation 5 starting from 4-nitro-1H-pyrazol-3-carboxylic acid (Sigma-Aldrich Chemical Co.)

### Preparation 17

### (3-Benzyloxy-phenyl)-acetic Acid Benzyl Ester

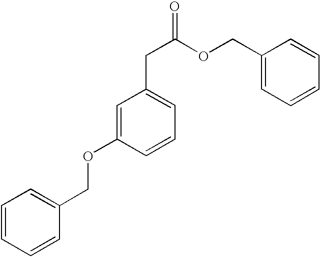

3-Hydroxy-phenyl-acetic acid (15.3 g, 101 mmol), benzyl bromide (36.2 g, 202 mmol) and potassium carbonate (29.2 g, 202 mmol) were suspended in dimethylformamide (300 ml) and the reaction was heated to reflux under nitrogen for 44 h. The reaction mixture was cooled, filtered and the filtrate was concentrated under reduced pressure. The residue was partitioned between ethyl acetate (200 ml) and water (200 ml), and the aqueous phase was extracted with ethyl acetate (2×200 ml). The combined organic extracts were washed with brine (200 ml), dried over Na2SO4 and the solvent was removed under reduced pressure. The residue was purified by flash column chromatography on silica gel eluting with pentane:ethyl acetate (95:5, by volume) to give the title product (10.7 g) as a white solid.

### Preparation 18

### (3-Benzyloxy-phenyl)-acetic Acid

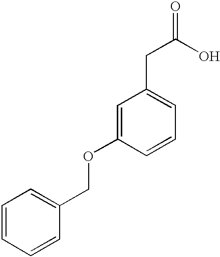

1N Sodium hydroxide solution (35 ml, 35 mmol) was added to a solution of the product from preparation 17 (5.3 g, 16 mmol) in methanol (350 ml) at room temperature under nitrogen. The reaction was heated to reflux for 2 h, and the solvent was removed under reduced pressure. The residue was dissolved in water (500 ml) and extracted with ether (3×350 ml). The aqueous phase was acidified to pH 1 with concentrated hydrochloric acid and the resultant precipitate was isolated by filtration and dried under vacuum to give the title product (3.08 g) as a white solid, m.p. 127-129° C.; ¹H NMR (400 MHz, CDCl3): δ=7.26-7.43 (5H, m), 7.20-7.26 (1H, m, partially masked by solvent), 6.84-6.96 (3H, m+s), 5.04 (2H, s), 3.62 (2H, s) ppm; LRMS (electrospray): m/z [M−H]+241; Anal. Found C, 74.21; H, 5.82. C15H14O requires C, 74.36; H, 5.82%.

### Preparation 19

### (4-Hydroxy-3-methoxy-phenyl)-acetic Acid Methyl Ester

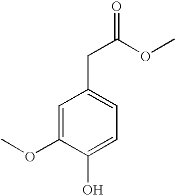

Concentrated sulfuric acid (12 ml) was added to a solution of (4-hydroxy-3-methyoxy-phenyl)-acetic acid (22.5 g, 123 mmol) in methanol (450 ml) at room temperature, and the reaction was heated to 90° C. for 2.45 h. The reaction was then cooled to room temperature and stirred for 18 h, and the solvent was removed under reduced pressure. The residue was suspended in ice water (300 ml) and extracted with diethylether (2×300 ml). The combined organic extracts were washed with sat. sodium bicarbonate solution (2×100 ml), brine (100 ml), dried over Na2SO4 and concentrated under reduced pressure. The residue was purified by flash column chromatography on silica gel eluting with a solvent gradient of cyclohexane:ethyl acetate (80:20 changing to 70:30, 60:40 and finally 1:1, by volume) to give the title product (23 g) as a yellow oil; ¹H NMR (400 MHz, CDCl₃): δ=6.82-6.85 (1H, d), 6.80 (1H, s), 6.76-6.79 (1H, d), 5.49 (1H, s), 3.86 (3H, s), 3.66 (3H, s), 3.53 (2H, s) ppm; LRMS (electrospray): m/z [M+Na]+219.

### Preparation 20

### (4-Cyclopentyloxy-3-methoxy-phenyl)-acetic Acid Methyl Ester

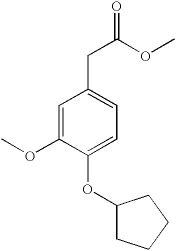

Cyclopentanol (7.7 ml, 85 mmol) and triphenylphosphine (28 g, 107 mmol) were added to a solution of the product from preparation 19 (14 g, 71 mmol) in tetrahydrofuran (280 ml) under nitrogen at 0° C. Diethylazodicarboxylate (15.7 ml, 100 mmol) was then added dropwise and the reaction was allowed to warm to room temperature and stirred for 44 h. The solvent was removed under reduced pressure, pentane (200 ml) was added and the suspension was filtered. The filtrate was concentrated under reduced pressure and purified by flash column chromatography on silica gel eluting with a solvent gradient of cyclohexane:ethyl acetate (90:10 changing to 85:15, by volume) to give the title product (12.4 g) as a colourless oil; ¹H NMR (400 MHz, CD₃OD): δ=6.79-6.85 (2H, m), 6.73-6.79 (1H, d), 4.73-4.79 (1H, brs), 3.79 (3H, s), 3.64 (3H, s), 3.53 (2H, s), 1.74-1.89 (6H, m), 1.56-1.67 (2H, m) ppm; LRMS (electrospray): m/z [M+Na]+287; Anal. Found C, 68.01; H, 7.74. C₁₅H₂₀O₄ requires C, 68.16; H, 7.63%.

### Preparation 21

### (4-Cyclopentyloxy-3-methoxy-phenyl)-acetic Acid

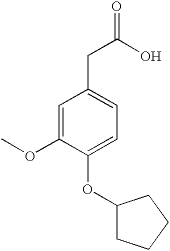

Sodium hydroxide (4.75 g, 119 mmol) was added to a solution of the product from preparation 20 (12.4 g, 46.9 mmol) in methanol (100 ml)/water (100 ml) and the reaction was stirred at room temperature for 3.5 h. The methanol was removed under reduced pressure and the aqueous phase was washed with diethylether (100 ml) then acidified to pH2 using concentrated hydrochloric acid. This was then extracted with ethyl acetate (2×200 ml) and the combined organic extracts were washed with brine (100 ml), dried over Na2SO4 and concentrated under reduced pressure to give the title product (11.1 g) as a white solid; 1H NMR (400 MHz, CD3OD): δ=6.87 (¹H, s), 6.81-6.86 (1H, d), 6.76-6.80 (1H, d), 4.75-4.79 (1H, brs), 3.78 (3H, s), 3.49 (2H, s), 1.71-1.89 (6H, m), 1.56-1.64 (2H, m) ppm; LRMS (electrospray): m/z [M−H]+249, [2M−H]+499; Anal. Found C, 67.15; H, 7.25. C₁₄H₁₈O₄ requires C, 67.18; H, 7.25%.

### Preparation 22

### 2,4-Dimethylphenyl-acetic Acid

2,4-Dimethylbenzylcyanide (70 g, 0.48 mol) was mixed with water (134 ml) and concentrated sulfuric acid (106 ml, 1.98 mol) was added slowly. The reaction was heated to reflux for 3 h, then cooled to room temperature over 18 h. The mixture was poured onto crushed ice (500 ml), stirred for 1 h and the resulting precipitate was isolated by filtration. After washing with water the solid was dissolved in 1.2M sodium hydroxide solution (500 ml), extracted with dichloromethane (2×250 ml) and the aqueous phase was treated with decolourising carbon (2 g) at reflux for 10 min and filtered hot through hyflo supercel. The filtrate was then acidified with concentrated hydrochloric acid and the resulting precipitate was isolated by filtration, washed with water and dried under vacuum to give the title product (52.6 g) as a white solid; 1H NMR (250 MHz, CD₃OD/D2O): δ=6.88-7.03 (3H, m), 3.48-3.68 (2H, s), 2.23 (6H, s) ppm.

### Preparation 23

### Benzene Sulfonic Acid 2-chloro-ethyl Ester

2-Chloroethanol (1168 g, 14.5 mol) and benzene sulfonyl chloride (2780 g, 15.7 mol) were stirred together at −5° C. and pyridine (2158 g, 27.2 mol) was added over a 3 h period, maintaining the temperature below 0° C. The reaction was stirred for a further 3 h at −5-0° C. and was then allowed to warm to room temperature over 18 h. After pouring into a mixture of ice (10 l) and water (10 l) the reaction was stirred for 15 min, extracted with ether (10 l) and the organic phase was washed with 5N HCl (2×2 l) and water (2×4 l). It was then dried over MgSO₄ and concentrated under reduced pressure to give the title product (1921 g) as an orange oil; ¹H NMR (250 MHz, CDCl₃): δ=7.78-8.02 (2H, m), 7.58-7.78 (3H, m), 4.20-4.45 (2H, t), 3.60-3.81 (2H, t) ppm.

### Preparation 24

### 2-Hydroxy-phenyl-acetic Acid Ethyl Ester

2-Hydroxy-phenyl-acetic acid (30.4 g, 0.2 mol) was dissolved in chloroform (200 ml) and thionyl chloride (50 ml, 0.2 mol) was added. The reaction was gently refluxed for 2 h, upon which the mixture was concentrated under reduced pressure. The residue was slowly poured into ethanol (200 ml) maintaining a temperature of 10-20° C. The solvent was removed under reduced pressure and the residue was purified by thermal distillation to give the title product (31.6 g) as a yellow oil, b.p. 146-150° C.; νₘₐₓ (thin film) 1710 cm−¹ (C═O, ester).

### Preparation 25

### [2-(2-Chloro-ethoxy)-phenyl]-acetic Acid Ethyl Ester

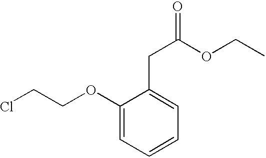

50% Sodium hydride in mineral oil (8.11 g, 169 mmol) was added portionwise to a solution of the product from preparation 24 (30.4 g, 169 mmol) in dimethylformamide (100 ml). After the initial effervescence had ended the reaction was heated to 100° C. for 10 min and was cooled to room temperature. A solution of benzene sulfonic acid 2-chloro-ethyl ester (37.2 g, 169 mmol) in dimethylformamide (5 ml) was then added and the reaction was heated to 100° C. for 1 h, and allowed to cool to room temperature over 18 h. The reaction mixture was partitioned between diethylether (300 ml) and water (300 ml) and the organic phase was removed and washed with water (100 ml), dried over MgSO₄ and the solvent was removed under reduced pressure. The residue was purified by thermal distillation to give the title product (22.0 g) as a pale yellow oil; b.p. 170-180° C.; νₘₐₓ (thin film) 1735 cm⁻¹ (C═O, ester); no O—H stretch; Anal. Found C, 59.35; H, 6.29. C₁₂H₁₅ClO₃ requires C, 59.38; H, 6.23%.

### Preparation 26

### [2-(2-Imidazol-1-yl-ethoxy)-phenyl]-acetic Acid Ethyl Ester

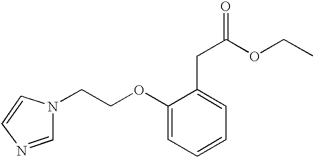

To a solution of imidazole (4.5 g) in dry dimethylformamide (80 ml) at room temperature was added sodium hydride (3.17 g of a 50% suspension). The mixture was heated at 100° C. for 10 mins then cooled to room temperature. The product from preparation 25 (16 g) was added in dry dimethylformamide (5 ml) and then heated to 100° C. for 4.5 hours. The reaction mixture was cooled and water was added. The mixture was extracted with chloroform, the combined extracts dried (MgSO₄) and the solvent removed to give the crude product. The crude product was converted to the hydrochloride salt and purified by recrystalisation from isopropyl alcohol/ethyl acetate to give the title product as its hydrochloride salt, m.p. 129.5-130.5° C.

### Preparation 27

### [2-(2-Imidazol-1-yl-ethoxy)-phenyl]-acetic Acid

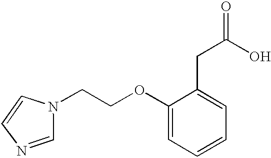

The product from Preparation 26 (3.5 g, 113 mmol) was stirred in 50% aqueous hydrochloric acid (20 ml) at 100° C. for 6 h. After cooling to room temperature the solvent was removed under reduced pressure and the residue was recrystallised from isopropyl alcohol to give the title product (2.73 g) as a white solid; m.p. 146-147° C.; νₘₐₓ (thin film) 3410 (O—H), 1722 cm⁻¹ (C═O, acid); Anal. Found C, 54.89; H, 5.25; N, 9.80. C₁₃H₁₄N₂O₃. 1 mol HCl requires C, 55.22; H, 5.35; N, 9.91%.

### Preparation 28

### 5-Cyclopentyl-4-[2-(2-trifluoromethoxy-phenyl)-acetylamino]-1H-pyrazole-3-carboxylic Acid

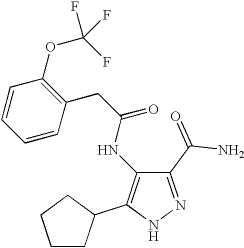

Carbonyldiimidazole (84 mg, 0.515 mmol) was added to a solution of 2-trifluoromethyoxyphenyl acetic acid (113 mg, 0.515 mmol) in tetrahydrofuran (4 ml) under nitrogen at room temperature, and the mixture was stirred for 3 h. The product from preparation 10 (100 mg, 0.515 mmol) was then added and the reaction was stirred for 18 h. The reaction mixture was diluted with water (20 ml), acidified to pH2 with 2N HCl and extracted with ethyl acetate (2×20 ml). The combined organic extracts were dried over MgSO₄ and concentrated under reduced pressure to give the title product (120 mg) as an off-white solid; LRMS (electrospray): m/z [M+H]+397, [M−H]+395.

Preparations 29 to 34 of general formula II were prepared by analogous procedures to Preparation 28 from the staring materials indicated in Table 7.

TABLE 7

| | |
|---|---|
| | (II) |
| 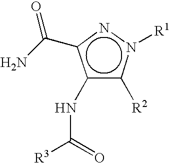 | |

| | | | | Starting | |
|---|---|---|---|---|---|
| Prep | R¹ | R² | R³ | pyrazole | Starting acid |
| 29 | H | isobutyl | 2-trifluoromethoxybenzyl | Prep 9 | 2-trifluoromethoxy |
| | | | | | phenyl acetic acid |
| 30 | H | 3-pyridyl | 2-trifluoromethoxybenzyl | Prep 13 | 2-trifluoromethoxy |
| | | | | | phenyl acetic acid |
| 31 | Me | isopropyl | 3-chlorobenzyl | Prep 11 | 3-chlorophenyl acetic |
| | | | | | acid |
| 32 | H | isopropyl | 2-(3-chlorobenzyloxy)-benzyl | Prep 6 | 2-(3- |
| | | | | | chlorobenzyloxy)phenyl |
| | | | | | acetic acid |
| 33 | H | isopropyl | 2-(4-chlorobenzyloxy)-benzyl | Prep 6 | 2-(4- |
| | | | | | chlorobenzyloxy)phenyl |
| | | | | | acetic acid |
| 34 | H | H | 2-trifluoromethoxybenzyl | Prep 15 | 2-trifluoromethoxyphenyl |
| | | | | | acetic acid |

### Preparation 35

### 4-{[(2-Benzyloxy-5-chloro-phenyl)acetyl]amino}-5-isopropyl-1H-pyrazole-3-carboxamide

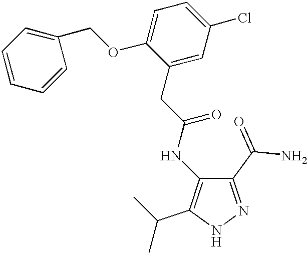

1-Propylphosphonic acid cyclic anhydride (0.39 ml of a 50% solution in ethyl acetate, 0.57 mmol) was added to a solution of 2-benzyloxy-5-chlorophenyl acetic acid (132 mg, 0.475 mmol), the product from preparation 6 (80 mg, 0.475 mmol) and triethyl amine (0.132 ml, 0.95 mmol) in dimethylformamide (4 ml) under nitrogen at room temperature and the reaction was stirred for 18 h. The reaction mixture was diluted with brine (20 ml) and extracted with ethyl acetate (2×20 ml). The combined organic extracts were dried over MgSO₄ and concentrated under reduced pressure to give the title product (191 mg) as an off-white solid; LCMS: m/z 427 [M+H]+.

### Preparation 36

### 5-(2-Trifluoromethoxybenzyl)-3-iodo-1,6-dihydro-pyrazolo[4,3-d]pyrimidin-7-one

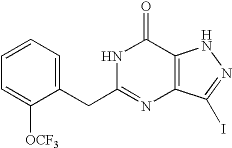

N-Iodosuccinimide (326 mg, 1.45 mmol) was added to a solution of the product from preparation 37 (300 mg, 0.967 mmol) in dry dimethylformamide (5 ml) at room temperature. The mixture was heated at 55° C. for 18 h, cooled and concentrated in vacuo. The residue was dissolved in ethyl acetate/tetrahydrofuran and washed with water and brine, dried (MgSO4), filtered and concentrated. The crude product was purified by flash chromatography (1→2% MeOH/CH₂Cl₂) to afford the title product (169 mg) as an off-white solid; ¹H NMR (400 MHz, CD₃OD) δ 7.35 (m, 4H); 4.1 (s, 2H); LCMS: m/z 437 [M+H]+.

### Preparation 37

### 5-(2-trifluoromethoxy-benzyl)-1,6-dihydro-pyrazolo[4,3-d]pyrimidin-7-one

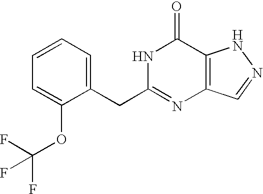

The title product was prepared by an analogous process to that of Example 3 starting from the product of preparation 34; MS: m/z [M+H]+311.

Assay

The ability of the compounds of the invention to inhibit the PDE9 enzyme was determined using the following in vitro assay. The assay used purified recombinant human PDE9.

The assay uses [3H]cGMP which is hydrolysed by the PDE9 enzyme to the 5′-nucleotide [3H]GMP. The [3H]GMP binds to yttrium silicate scintillation proximity assay (SPA) beads, and detected by scintillation counting. Inhibition of activity is determined relative to the activity of uninhibited controls.

Materials/Reagents

Recombinant human PDE-9 enzyme (Flag-tagged) was obtained by expression in a baculovirus/Sf9 cell system and purified by anti-FLAG monoclonal antibody affinity chromatography (D A Fisher, J F Smith, J S Pillar, S H Denis, J B Cheng (1998), J. Biol. Chem., 273: 15559-15564).

Phosphodiesterase Scintillation Proximity Assay (SPA) beads (Yttrium Silicate) were obtained from Amersham Biotech.

[³H]Guanosine 3′,5′-Cyclic Phosphate ([³H]-cGMP), Ammonium Salt was obtained from Amersham Biotech.

Preparation of Assay Buffers and Solutions

Buffer A was prepared containing Tris.HCl (20 mM), MgCl₂.6H₂O (5 mM) in water. The resulting solution was used at 30 oC. and had a pH of pH=7.4.

Buffer B was prepared containing Bovine Serum Albumin (2 mg/ml) (BSA) in Buffer A. It was prepared fresh and filter sterilised.

PDE9 enzyme solution was prepared in Buffer B (dilution factor determined such that no more than 30% breakdown of substrate occurred, but typically 1:35,000).

cGMP substrate was prepared from a 50 nM stock of guanosine 3′:5′-cyclic monophosphate (cGMP) prepared to give final assay concentration of 25 nM (to prepare 5 ml based on specific activity of labelled substrate of 16.0 Ci/mmol; 1 mCi/ml, add 4 μl [³H]cGMP to 4.996 ml Buffer A).

SPA beads were prepared by creating a suspension of beads in water (20 mg/ml) (28 ml per pack) containing 3 mM cold cGMP to effectively quench the reaction.

Preparation of Compounds

The compounds of the invention were diluted by a factor of 50 (i.e. 2 μl in 100 μl) when constituted in the final assay mix. Compound stock was prepared at 4 mM in DMSO. Dilute 1/8 with DMSO to give 500 μM solutions.

A 4 mM stock of standard inhibitor was prepared in DMSO. The standard inhibitor chosen was 5-(3-bromobenzyl)-3-n-propyl-1,6-dihydro-7H-pyrazolo[4,3-d]pyrimidin-7-one. The solution was further diluted with DMSO to give a 500 μM solution.

For 10-point ½ log dilution, 200 μl of compound and standard solutions were dispensed into a 96-well V-bottom plate and the compounds further diluted with DMSO in steps of 1:3.16. Following serial dilution, 2 μl of compound dilutions were dispensed in duplicate into microtitre plates with 2 μl DMSO added to controls as shown below.

| Control | Blank | Test compound dilution |
|---|---|---|
| 2 μl DMSO | 2 μl DMSO | 2 μl Test compound |
| 25 μl Buffer A | 25 μl Buffer A | 25 μl Buffer A |
| 25 μl Enzyme | 25 μl Buffer B | 25 μl Enzyme |
| 50 μl Substrate | 50 μl Substrate | 50 μl Substrate |
| 50 μl SPA to stop | 50 μl SPA to stop | 50 μl SPA to stop |

Assay Procedure

To the microtitre plates containing solutions of compounds of the invention (2 μl) was added Buffer A (25 μl) to all wells. Buffer B (25 μl) was added to blank wells. Enzyme solution (25 μl) was added all wells except blanks. Substrate solution (50 μl) was added to each well. The plates were sealed and incubated for 15 minutes on a plate-shaker at 30 oC. SPA bead solution (50 μl) was added (containing excess cGMP) to each well to stop the reaction (Note: yttrium silicate beads are dense and require constant agitation whilst being added to each plate). The plates were shaken for 15 minutes to allow the beads to bind the GMP product and then allowed to settle for 30 minutes.

The plates were then analysed using a scintillation counter (such as the NXT TopCount)TM.

For each compound the percentage inhibition was calculated by the following equation:

(mean maximum−compound value)/(mean maximum−mean minimum)×100

IC50 values were determined from sigmoid dose response curves of enzyme activity vs. compound concentration. The IC50 of the standard was expected to fall in the range 30-50 nM.

The ability of the compounds to inhibit the PDE1 enzyme was determined by an assay procedure broadly analogous to that described above for PDE9. The differences are as follows: i) human PDE1 enzyme was isolated from human ventricle homogenate by chromatographic separation of a high speed centrifugation supernatant fraction; ii) the cGMP substrate (1 μM) was prepared by combining 0.436 ml of a 10 μM stock of unlabelled guanosine 3′:5′-cyclic monophosphate (cGMP) with 10 μM [3H]cGMP (15.6 Ci/mmol) and 4.554 ml Buffer A; the final assay concentration of cGMP in the assay being 0.5 μM, iii) the PDE1 inhibitor used as a standard was 5-[4-(N,N-diethylamino)benzyl]-1-methyl-3-n-propyl-1,6-dihydro-7H-pyrazolo[4,3-d]pyrimidin-7-one, and iv) during the procedure the plates were incubated for 30 minutes.

The compounds of the invention were tested using the above assays and found to inhibit the PDE9 enzyme.

Compounds 1-21, 23-31, 33-77, 118, 127-208, 213, 214, 224 and 256-263 were found to have a greater than 40% inhibition against PDE9 at a concentration of 1 μM.

In particular, compound 52 was found to have an IC50 against PDE9 of 126 nM; compound 204 was found to have an IC50 against PDE9 of 143 nM; and compound 258 was found to have an IC50 against PDE9 of 141 nM. Compounds 52, 204 and 258 were all greater than 10 fold selective for PDE9 over PDE1.

1. A compound of formula I, a pharmaceutically acceptable salt, solvate or prodrug thereof 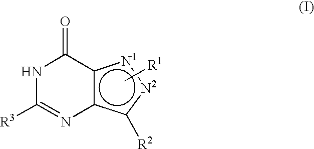
wherein
R¹ is H or C₁₋₆ alkyl, wherein R¹ is attached to either N¹ or N²;
R² is C₁₋₆ alkyl optionally substituted by hydroxy or alkoxy; C₃₋₇ cycloalkyl optionally substituted by alkyl, hydroxy or alkoxy; a saturated 5-6-membered heterocycle optionally substituted by alkyl, hydroxy or alkoxy; het¹ or Ar¹;
R³ is C₁₋₆ alkyl optionally substituted by 1 or 2 groups independently selected from: Ar²; C₃₋₇cycloalkyl optionally substituted by C₁₋₆alkyl; OAr²; SAr²; NHC(O)C₁₋₆ alkyl; het²; xanthene; and naphthalene;
wherein Ar¹ and Ar² are independently groups of formula 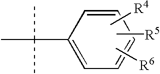
wherein R⁴, R⁵ and R⁶ are independently selected from: hydrogen, halo, phenoxy, phenyl, CF₃, OCF₃, R⁷, SR⁷ and OR⁷, wherein R⁷ is C₁₋₆ alkyl optionally substituted by het³ or by a phenyl group optionally substituted by 1, 2 or 3 groups independently selected from halo, CF₃, OCF₃, C₁₋₆ alkyl and C₁₋₆ alkoxy; or wherein R⁴ and R⁵ combine to form a 3 or 4 atom link, wherein said link may incorporate one or two heteroatoms independently selected from O, S and N; and
wherein het¹, het² and het³, which may be the same or different, are aromatic 5-6 membered heterocycles containing 1, 2 or 3 heteroatoms, independently selected from O, S and N, said heterocycle optionally substituted by 1, 2 or 3 substituents, independently selected from C₁₋₆ alkyl, C₁₋₆alkoxy, halo and phenyl optionally substituted by 1, 2 or 3 groups independently selected from halo and C₁₋₆ alkyl;
with the provisos that when
a) R¹ is attached to N¹, R¹ is C₁₋₃ alkyl and R² is propyl then R³ is not methyl substituted by Ar¹, and
b) R¹ is attached to N¹, R¹ is C₁₋₆ alkyl and R² is methyl then R³ is not C₁₋₄alkyl substituted by Ar¹.

2 A compound according to claim 1 wherein the compound is of formula Ia, a pharmaceutically acceptable salt, solvate or prodrug thereof 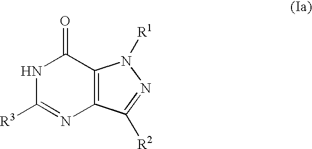
wherein
R¹ is H or C₁₋₆ alkyl;
R² is C₁₋₆ alkyl optionally substituted by hydroxy or alkoxy; C₃₋₇ cycloalkyl optionally substituted by alkyl, hydroxy or alkoxy; a saturated 5-6-membered heterocycle optionally substituted by alkyl, hydroxy or alkoxy; het¹ or Ar¹;
R³ is C₁₋₆ alkyl optionally substituted by 1 or 2 groups independently selected from: Ar²; C₃₋₇cycloalkyl optionally substituted by C₁₋₆alkyl; OAr²; SAr²; NHC(O)C₁₋₆ alkyl; het²; xanthene; and naphthalene;
wherein Ar¹ and Ar² are independently groups of formula 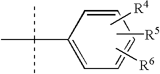
wherein R⁴, R⁵ and R⁶ are independently selected from: hydrogen, halo, phenoxy, phenyl, CF₃, OCF₃, R⁷, SR⁷ and OR⁷, wherein R⁷ is C₁₋₆ alkyl optionally substituted by het³ or by a phenyl group optionally substituted by 1, 2 or 3 groups independently selected from halo, CF₃, OCF₃, C₁₋₆ alkyl and C₁₋₆alkoxy; or wherein R⁴ and R⁵ combine to form a 3 or 4 atom link, wherein said link may incorporate one or two heteroatoms independently selected from O, S and N; and
wherein het¹, het² and het³, which may be the same or different, are aromatic 5-6 membered heterocycles containing 1, 2 or 3 heteroatoms, independently selected from O, S and N, said heterocycle optionally substituted by 1, 2 or 3 substituents, independently selected from C₁₋₆ alkyl, C₁₋₆alkoxy, halo and phenyl optionally substituted by 1, 2 or 3 groups independently selected from halo and C₁₋₆ alkyl;
with the provisos that when
a) R¹ is C₁₋₃ alkyl and R₂ is propyl then R³ is not methyl substituted by Ar¹, and
b) R¹ is C₁₋₆ alkyl and R² is methyl then R³ is not C₁₋₄alkyl substituted by Ar¹.

3. A compound according to any preceding claim wherein R¹ is hydrogen or CH₃.

4. A compound according to any preceding claim wherein R¹ is hydrogen.

5. A compound according to any preceding claim wherein R² is C₃₋₄ alkyl, cyclopentyl or pyridyl.

6. A compound according to any preceding claim wherein R² is 3-pyridyl.

7. A compound according to any preceding claim wherein R³ is C₁₋₃ alkyl optionally substituted by 1 or 2 groups independently selected from: Ar²; C₃₋₇ cycloalkyl optionally substituted by C₁₋₆alkyl; and het².

8. A compound according to any preceding claim wherein R³ is C₁₋₃ alkyl optionally substituted by Ar².

9. A compound according to any preceding claim wherein R³ is methyl substituted by Ar₂.

10. A compound according to any preceding claim wherein R⁴, R⁵ and R⁶ are independently selected from: hydrogen, halo, phenoxy, phenyl, CF₃, OCF₃, R⁷, SR⁷, and OR⁷, wherein R⁷ is C₁₋₆alkyl optionally substituted by a het³ group or by a phenyl group optionally substituted by 1, 2 or 3 groups independently selected from halo, CF₃, OCF₃, C₁₋₆ alkyl and C₁₋₆alkoxy; or wherein R⁴ and R⁵ combine to form a 3 atom link wherein said link contains an oxygen atom.

11. A compound according to any preceding claim wherein R⁴, R⁵ and R⁶ are independently selected from hydrogen, halo, CF₃, OCF₃, phenoxy, and OC₁₋₆ alkyl optionally substituted by phenyl optionally substituted by halo, CF₃, OCF₃ or C₁₋₆ alkyl.

12. A compound according to any preceding claim wherein R⁴, R⁵ and R⁶ are independently selected from hydrogen, chloro, OCF₃, CF₃, phenoxy and OC₁₋₆ alkyl substituted by phenyl.

13. A compound according to any preceding claim wherein R⁴, R⁵ and R⁶ are independently selected from hydrogen, chloro, OCF₃ and OC₁₋₃ alkyl substituted by phenyl.

14. A compound according to any one of claims 1 to 7 wherein het² is an aromatic 5-6 membered heterocycle containing 1 or 2 nitrogen atoms optionally containing a further heteroatom, said heterocycle being optionally substituted by 1, 2 or 3 substituents, each independently selected from C₁₋₆ alkyl, halo and phenyl optionally substituted by 1, 2 or 3 groups independently selected from halo and C₁₋₆ alkyl.

15. A compound according to claim 14 wherein het² is an aromatic 5-membered heterocycle containing 1 or 2 nitrogen atoms (optionally containing a further heteroatom) said heterocycle being optionally substituted by 1 substituent selected from C₁₋₆ alkyl, halo and phenyl optionally substituted by 1, 2 or 3 groups independently selected from halo and C₁₋₆ alkyl.

16. A compound according to claim 15 wherein het² is an aromatic 5 membered heterocycle containing 1 or 2 nitrogen atoms (optionally containing a further heteroatom) and optionally substituted by phenyl optionally substituted by halo.

17. A compound according to claim 16 wherein het² is imidazolyl or oxadiazolyl.

18. A compound according to claim 1 wherein the compound is:
5-(3-chlorobenzyl)-3-isopropyl-1,6-dihydro-pyrazolo[4,3-d]pyrimidine-7one (compound 1);
3-isopropyl-5-(2-phenoxybenzyl)-1,6-dihydro-pyrazolo[4,3-d]pyrimidine-7-one (compound 52);
3-(3-pyridinyl)-5-(2-benzyloxybenzyl)-1,6-dihydro-pyrazolo[4,3-d]pyrimidine-7-one (compound 138);
3-isopropyl-5-(2-trifluoromethoxybenzyl)-1,6-dihydro-pyrazolo[4,3-d]pyrimidine-7-one (compound 156);
3-cyclopentyl-5-(2-benzyloxybenzyl)-1,6-dihydro-pyrazolo[4,3-d]pyrimidine-7-one (compound 204);
3-(3-pyridinyl)-5-(2-trifluoromethylbenzyl)-1,6-dihydro-pyrazolo[4,3-d]pyrimidine-7-one (compound 215);
3-cyclopentyl-5-(2-trifluoromethoxy-benzyl)-1,6-dihydro-pyrazolo[4,3-d]pyrimidin-7-one (compound 258); and
3-(3-pyridinyl)-5-(2-trifluoromethoxybenzyl)-1,6-dihydro-pyrazolo[4,3-d]pyrimidin-7-one (compound 260).

19. The use of a compound defined in any preceding claim, a pharmaceutically acceptable salt or solvate thereof, in the manufacture of a medicament for treating or preventing a cardiovascular disorder, disease or condition.

20. The use according to claim 19, a pharmaceutically acceptable salt or solvate thereof, wherein the disorder, disease or condition is systemic hypertension.

21. A compound defined in any one of claims 1 to 18, a pharmaceutically acceptable salt or solvate thereof, for use as a medicament.

22. A pharmaceutical composition comprising a compound defined in any one of claims 1 to 18, a pharmaceutically acceptable salt or solvate thereof, together with a pharmaceutically acceptable excipient, diluent or carrier.

23. A process for preparing a compound of formula I as defined in any one of claims 1 to 18 comprising reacting a compound of formula II with a suitable reagent to effect cyclisation.

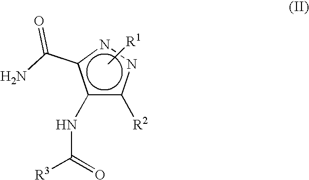

24. The use of a PDE9 inhibitor in the manufacture of a medicament for treating or preventing a cardiovascular disorder, disease or condition.

25. The use according to claim 24 wherein the PDE9 inhibitor has a greater than 40% inhibition against PDE9 at a concentration of 1 micromolar.

26. The use according to claim 25 wherein the PDE9 has a selectivity for PDE9 over PDE1 of greater than 10.
